(19) United States
(12) Patent Application Publication
(10) Pub. No.: US 2026/0130644 A1
(43) Pub. Date: May 14, 2026

(54) ELECTRICAL CONNECTIONS FOR IVUS CATHETERS

(71) Applicant: Boston Scientific Scimed, Inc., Maple Grove, MN (US)

(72) Inventors: Robert Fagerlund, White Bear Lake, MN (US); Andrew Brian Graveley, Shoreview, MN (US); Jan Seppala, Loretto, MN (US); Phillip John Poisson, Maple Grove, MN (US); Cole Thomas Theobald, Golden Valley, MN (US); Kasey Sontida Mohlke, Lakeville, MN (US)

(73) Assignee: Boston Scientific Scimed, Inc., Maple Grove, MN (US)

(21) Appl. No.: 19/382,422

(22) Filed: Nov. 7, 2025

Related U.S. Application Data
(60) Provisional application No. 63/718,037, filed on Nov. 8, 2024.

(51) Int. Cl. A61B 8/00; A61B 8/08; A61B 8/12

(52) U.S. Cl. CPC A61B 8/445; A61B 8/0891; A61B 8/12

(57) ABSTRACT

An intravascular imaging device may include a shaft with proximal and distal end regions, and an imaging assembly movably disposed within the shaft. The imaging assembly may comprise a drive cable, a housing coupled to the drive cable's distal end, a transducer within the housing, and a coaxial cable extending through the drive cable and electrically coupled to the transducer. The coaxial cable may be electrically coupled to a connection point on an upper surface of the transducer and electrically coupled to the housing. The device may include different configurations of the coaxial cable, such as a first member electrically coupled to the connection point and a second member electrically coupled to the housing. The housing may have various regions, including a cavity for receiving the transducer.

### CROSS-REFERENCE TO RELATED APPLICATIONS

This application claims the benefit of priority under 35 U.S.C. § 119 of U.S. Provisional Application No. 63/718,037, filed Nov. 8, 2024, the entire disclosure of which is hereby incorporated by reference.

### TECHNICAL FIELD

The present disclosure pertains to medical imaging, and systems and methods for medical imaging. More particularly, the present disclosure pertains to distal tip designs of intravascular ultrasound catheters.

### BACKGROUND

A wide variety of medical imaging systems and methods have been developed for medical use, for example, use in imaging vascular anatomy. Some of these systems and methods include intravascular imaging modalities. These systems and methods include various configurations and may operate or be used according to any one of a variety of methods. Of the known vascular imaging systems and methods, each has certain advantages and disadvantages. Accordingly, there is an ongoing need to provide alternative systems and methods for vascular imaging and assessment.

### BRIEF SUMMARY

This disclosure provides design, material, manufacturing method, and use alternatives for medical devices. An intravascular imaging system is disclosed.

In an example, an intravascular imaging device may include a shaft having proximal and distal end regions, and an imaging assembly movably disposed within the shaft. The imaging assembly may include a drive cable, a housing coupled to a distal end of the drive cable, wherein an intermediate region includes a generally planar inner surface extending at a non-parallel angle relative to a longitudinal axis of the imaging assembly, a transducer disposed within the housing, and a coaxial cable extending through the drive cable and electrically coupled to the transducer. The coaxial cable may be electrically coupled to a connection point on an upper surface of the transducer and electrically coupled to the housing.

Alternatively or additionally to any of the examples above, in another example, the connection point on the upper surface of the transducer may be a pre-tin solder.

Alternatively or additionally to any of the examples above, in another example, a first member of the coaxial cable may be electrically coupled to the connection point.

Alternatively or additionally to any of the examples above, in another example, a distal end region of the first member may be bent to align the distal end region of the first member with the connection point.

Alternatively or additionally to any of the examples above, in another example, a second member of the coaxial cable may be electrically coupled to the housing.

Alternatively or additionally to any of the examples above, in another example, the first and second members of the coaxial cable may be separated by a dielectric layer.

Alternatively or additionally to any of the examples above, in another example, the second member of the coaxial cable may be electrically coupled to the housing with an electrically conductive adhesive or solder.

Alternatively or additionally to any of the examples above, in another example, the housing may comprise a generally tubular proximal end region and a distal end region having a substantially solid cross-section, the intermediate region disposed between the proximal end region and the distal end region and having a cavity for receiving the transducer.

Alternatively or additionally to any of the examples above, in another example, the distal end region of the housing may further comprise a notch that may be circumferentially aligned with an active surface of the transducer.

Alternatively or additionally to any of the examples above, in another example, the device may further comprise a non-conductive potting adhesive disposed over an exposed portion of a first member of the coaxial cable.

Alternatively or additionally to any of the examples above, in another example, the proximal end region of the housing may include a cut-out region extending through a sidewall of the housing, the cut-out region extending proximally from the intermediate region of the housing.

Alternatively or additionally to any of the examples above, in another example, the transducer may comprise a backing layer extending from a proximal end to a distal end, the backing layer comprising at least a first portion having a first length and a second portion having a second length, the first portion extending proximally beyond a proximal end of the second portion to define a ledge; a piezoelectric layer disposed on the backing layer; and a matching layer disposed on the piezoelectric layer, the matching layer extending across an upper surface of the piezoelectric layer, the ledge of the backing layer, and at least some of the lateral and axial sides of the piezoelectric layer and the backing layer.

Alternatively or additionally to any of the examples above, in another example, a distal end region of a first portion of the coaxial cable may be positioned on the ledge of the backing layer.

Alternatively or additionally to any of the examples above, in another example, a region of the matching layer may be removed to define the connection point adjacent to the proximal end of the transducer.

Alternatively or additionally to any of the examples above, in another example, the device may further comprise an electrically conductive adhesive extending between the connection point and the ledge of the backing layer, the electrically conductive adhesive configured to electrically couple the connection point with the coaxial cable.

Alternatively or additionally to any of the examples above, in another example, the device may further comprise solder extending between the connection point and the ledge of the backing layer, the solder configured to electrically couple the connection point with the coaxial cable.

In an example, an intravascular imaging device may include a shaft having proximal and distal end regions, and an imaging assembly movably disposed within the shaft. The imaging assembly may include a drive cable, a housing coupled to a distal end of the drive cable, a transducer disposed within the housing, and a coaxial cable extending through the drive cable and electrically coupled to the transducer. The coaxial cable may be electrically coupled to a connection point on an upper surface of the transducer and electrically coupled to the housing.

Alternatively or additionally to any of the examples above, in another example, the connection point on the upper surface of the transducer may be a pre-tin solder.

Alternatively or additionally to any of the examples above, in another example, a first member of the coaxial cable may be electrically coupled to the connection point.

Alternatively or additionally to any of the examples above, in another example, a distal end region of the first member may be bent to align the distal end region of the first member with the connection point.

Alternatively or additionally to any of the examples above, in another example, a second member of the coaxial cable may be electrically coupled to the housing.

Alternatively or additionally to any of the examples above, in another example, the first and second members of the coaxial cable may be separated by a dielectric layer.

Alternatively or additionally to any of the examples above, in another example, the second member of the coaxial cable may be electrically coupled to the housing with an electrically conductive adhesive or solder.

Alternatively or additionally to any of the examples above, in another example, the housing may comprise a generally tubular proximal end region, an intermediate region having a cavity for receiving the transducer, and a distal end region having a substantially solid cross-section.

Alternatively or additionally to any of the examples above, in another example, the distal end region of the housing may further comprise a notch that may be circumferentially aligned with an active surface of the transducer.

Alternatively or additionally to any of the examples above, in another example, the device may further comprise a non-conductive potting adhesive disposed over an exposed portion of a first member of the coaxial cable.

Alternatively or additionally to any of the examples above, in another example, the proximal end region of the housing may include a cut-out region extending through a sidewall of the housing. The cut-out region may extend proximally from the intermediate region of the housing.

In an example, an intravascular imaging device may include a shaft having proximal and distal end regions, and an imaging assembly movably disposed within the shaft. The imaging assembly may include a drive cable, a housing coupled to a distal end of the drive cable, the housing comprising a proximal end region, an intermediate region having a cavity for receiving a transducer, and a distal end region having a substantially solid cross-section. A transducer may be disposed within the cavity of the housing, the transducer comprising a backing layer extending from a proximal end to a distal end, the backing layer comprising at least a first portion having a first length and a second portion having a second length, the first portion extending proximally beyond a proximal end of the second portion to define a ledge; a piezoelectric layer disposed on the backing layer; a matching layer disposed on the piezoelectric layer, the matching layer extending across an upper surface of the piezoelectric layer, the ledge of the backing layer, and at least some of the lateral and axial sides of the piezoelectric layer and the backing layer; and a coaxial cable extending through the drive cable and electrically coupled to the transducer.

Alternatively or additionally to any of the examples above, in another example, a distal end region of a first portion of the coaxial cable may be positioned on the ledge of the backing layer.

Alternatively or additionally to any of the examples above, in another example, a region of the matching layer may be removed to define a connection point adjacent to the proximal end of the transducer.

Alternatively or additionally to any of the examples above, in another example, the device may further comprise an electrically conductive adhesive extending between the connection point and the ledge of the backing layer. The electrically conductive adhesive may be configured to electrically couple the connection point with the coaxial cable.

In an example, an intravascular imaging device may include a shaft having proximal and distal end regions, and an imaging assembly movably disposed within the shaft. The imaging assembly may include a drive cable, a housing coupled to a distal end of the drive cable, a transducer disposed within the housing, a coaxial cable extending through the drive cable and electrically coupled to the transducer, and a connector assembly coupled to a proximal end of the drive cable. The connector assembly may include a hub having a cavity, a printed circuit board disposed within the cavity, and an electrically conductive connector shaft coupled to the drive cable and electrically connected to the printed circuit board.

Alternatively or additionally to any of the examples above, in another example, the electrically conductive connector shaft may comprise a proximal end region disposed over a distal end region of the hub.

Alternatively or additionally to any of the examples above, in another example, the electrically conductive connector shaft may comprise a distal end region secured to an outer surface of the drive cable.

Alternatively or additionally to any of the examples above, in another example, the device may further comprise one or more pogo pins extending from the printed circuit board and electrically coupling the printed circuit board with the connector shaft.

Alternatively or additionally to any of the examples above, in another example, the connector shaft may comprise a first inner diameter adjacent to a proximal end and a second, smaller inner diameter adjacent to a distal end.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1A is a partially cut away side view of an example medical device;

FIG. 1B is a side view of the example medical device of FIG. 1A;

FIG. 2 is a side view of a distal end region of an illustrative imaging assembly;

FIG. 3 is a cross-sectional view of the distal end region of the imaging assembly of FIG. 2;

FIG. 4 is a cross-sectional view of an illustrative housing and transducer assembly;

FIG. 5 is a perspective view of the illustrative housing of FIG. 4;

FIG. 6 is a side view of the illustrative housing of FIG. 5;

FIG. 7 is a cross-sectional view of the illustrative housing, taken at line 7-7 of FIG. 5;

FIG. 8 is an illustrative flow chart of a method for assembling an imaging assembly;

FIG. 9A is a perspective view of another illustrative housing;

FIG. 9B is a cross-sectional view of the illustrative housing, taken at line 9B-9B of FIG. 9A;

FIG. 10 is a perspective schematic view of an illustrative coaxial cable;

FIG. 11 is a perspective view of another illustrative housing;

FIG. 12 is a perspective view of the illustrative housing of FIG. 11 rotated clockwise by about 90° relative to FIG. 11;

FIG. 13 is a partial cross-sectional view of the illustrative housing of FIG. 11 with a drive cable, twisted pair cable, and transducer;

FIG. 14 is a cross-sectional view of another illustrative housing;

FIG. 15 is a perspective view of an illustrative transducer;

FIGS. 16-19 illustrate sequential steps of an illustrative method and mechanism for electrically coupling a transducer with a housing;

FIGS. 20-23 illustrate partial cross-sectional views of illustrative distal end regions of an imaging assembly which include alternative shapes and configurations of the coaxial cable;

FIG. 24 is a side view of an alternative configuration for a coaxial cable;

FIG. 25 is a schematic cross-sectional view of another illustrative housing and transducer assembly; and

FIG. 26 is a partial cross-sectional view of a hub that is disposed within the connector assembly and coupled with the drive cable.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, or characteristics. However, such recitations do not necessarily mean that all embodiments include the particular features, structures, or characteristics. Additionally, when particular features, structures, or characteristics are described in connection with one embodiment, it should be understood that such features, structures, or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Rotational intravascular ultrasound (IVUS) may be used for high-resolution imaging of the blood vessels during percutaneous coronary interventions (PCI). Increasing adoption of intravascular imaging for PCI may increase production volumes of IVUS catheters. Current IVUS catheters may require hand-assembly of microscopic components. This manual approach may be highly dependent on operator technique which may significantly impact production yields. The present disclosure is directed towards distal tip designs to increase manufacturability while maintaining device performance. While the present disclosure is described with respect to intravascular imaging, the devices and methods described herein can be used for pulmonary procedures/imaging or in other anatomy, as desired.

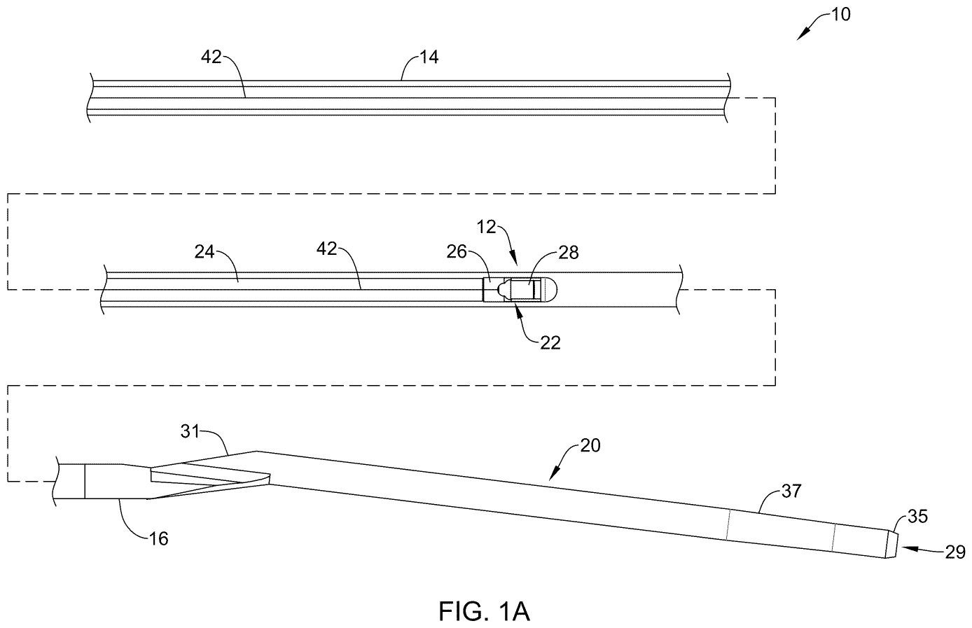

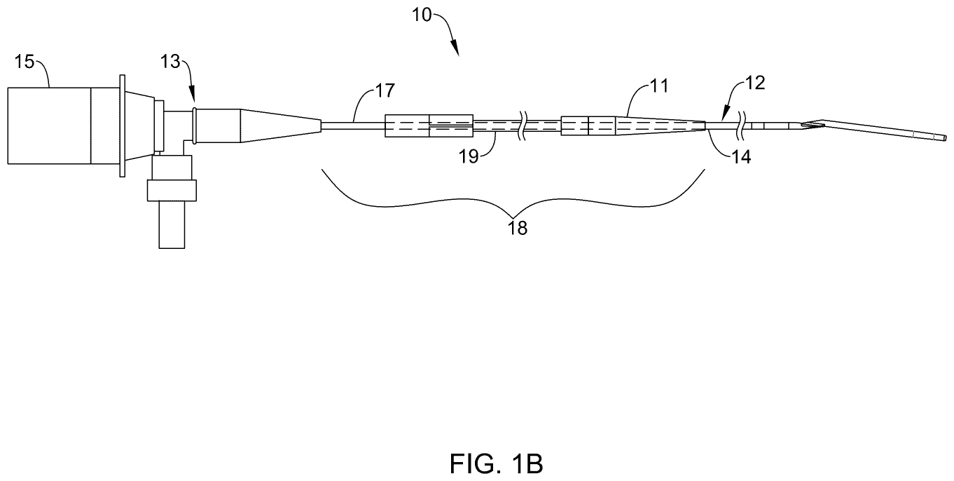

FIG. 1A is a side view of an example medical device 10. In at least some instances, the medical device 10 takes the form of an imaging medical device. For example, the medical device 10 may be an IVUS device that may be used to image a blood vessel. The structure/form of the medical device 10 can vary. In some instances, the medical device 10 may include an elongate shaft 12 having a proximal end region 14 and a distal end region 16. A tip member 20 may be coupled to or otherwise disposed adjacent to the distal end region 16 of the elongate shaft 12. The tip member 20 may include a guidewire lumen 29 having a guidewire exit port 31, an atraumatic distal end 35, one or more radiopaque markers 37, and/or other features. In some embodiments, the tip member 20 may extend at a non-parallel angle to the proximal end region 14 of the elongate shaft 12.

An imaging assembly 22 may be movably disposed within a lumen of the shaft 12. In general, the imaging assembly may be used to capture/generate images of a blood vessel. In some instances, the medical device may include devices and/or features similar to those disclosed in U.S. Patent Application Pub. No. 2012/0059241 and U.S. Patent Application Pub. No. 2017/0164925, the entire disclosures of which are herein incorporated by reference. In at least some instances, the medical device 10 may resemble and/or include features that resemble the OPTICROSS™ Imaging Catheter or the OPTICROSS™ HD Imaging Catheter, commercially available from BOSTON SCIENTIFIC, Marlborough, MA.

The imaging assembly 22 may include a drive cable or shaft 24, a housing 26, and an imaging member or transducer 28 coupled to the drive cable 24 and/or housing 26. In at least some instances, the transducer 28 includes an ultrasound transducer. Other transducers are also contemplated. The transducer 28 may be rotatable and/or axially translatable relative to the shaft 12. In order to do so, the shaft 12 may be connected to a control unit such as a motor drive unit. Illustrative drive motor units are described in U.S. Patent Application Pub. No. 2024/0325713, the entire disclosure of which is herein incorporated by reference. For example, the drive cable 24 may be rotated and/or translated in order to rotate and/or translate the transducer 28 (and the housing 26). A conductor 42 may be coupled to the transducer 28 and extend proximally therefrom. In some instances, the conductor 42 may take the form of a wire or cable (e.g., a coaxial cable) with suitable electrical conduction properties that allow the conductor 42 to energize the transducer 28.

The proximal end region 14 of the elongate shaft 12 may be coupled to a telescoping assembly 18 as shown in FIG. 1B. In general, the telescoping assembly 18 may be configured to allow a medical device operator to move the drive shaft 24 including the imaging core 22 proximally and distally within the catheter (e.g., relative to the elongate shaft 12), without having to move the entire catheter within the patient. This allows the catheter operator to easily change the location of the imaging core 22 within the patient. For example, the telescoping assembly 18 may be actuated to change the location of the imaging core 22 within the elongate shaft 12. An illustrative telescoping section is described in commonly assigned U.S. Patent Application Pub. No. 2023/0309962, the disclosure of which is herein incorporated by reference.

The proximal end region 14 of the elongate shaft 12 may be coupled to the telescoping assembly 18. For example, the proximal end region 14 of the elongate shaft 12 may be coupled to a distal hub 11 of the telescoping assembly 18. A proximal hub 13 may be coupled to the telescoping assembly 18 (e.g., at the proximal end of the telescoping assembly 18). The drive shaft 24 (see FIG. 1) may extend through the telescoping assembly 18 and be coupled to and/or otherwise secured to the proximal hub 13. The proximal hub 13 may include a connector assembly 15. In general, the connector assembly 15 may allow the medical device 10 (e.g., the elongate shaft 12) to connect to a control unit (e.g., a motor drive unit and/or the like).

The telescoping assembly 18 may include a first sheath 17 and a second sheath 19. In some instances, the first sheath 17 may be understood to be an inner telescoping tube 17 and the second sheath 19 may be understood to be an outer telescoping tube 19. Generally, the outer telescoping tube 19 may be disposed over the inner telescoping tube 17. The inner telescoping tube 17 may be coupled or otherwise secured to the proximal hub 13. The outer telescoping tube 19 may be coupled or otherwise secured to the distal hub 11. The inner telescoping tube 17 may be axially and/or rotatably moveable relative to the outer telescoping tube 19. Because the drive shaft 24 may be secured to the proximal hub 13 and because the elongate shaft 12 may be secured to the distal hub 11, movement of the proximal hub 13 relative to the distal hub 11 results in movement of the inner telescoping tube 17 and the drive shaft 24 relative to the distal hub 11 and/or the elongate shaft 12.

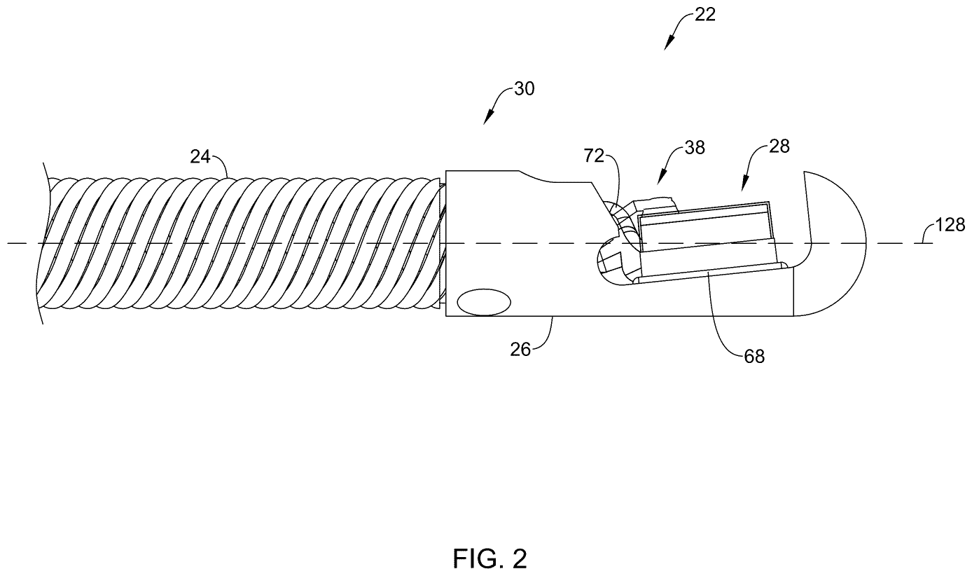

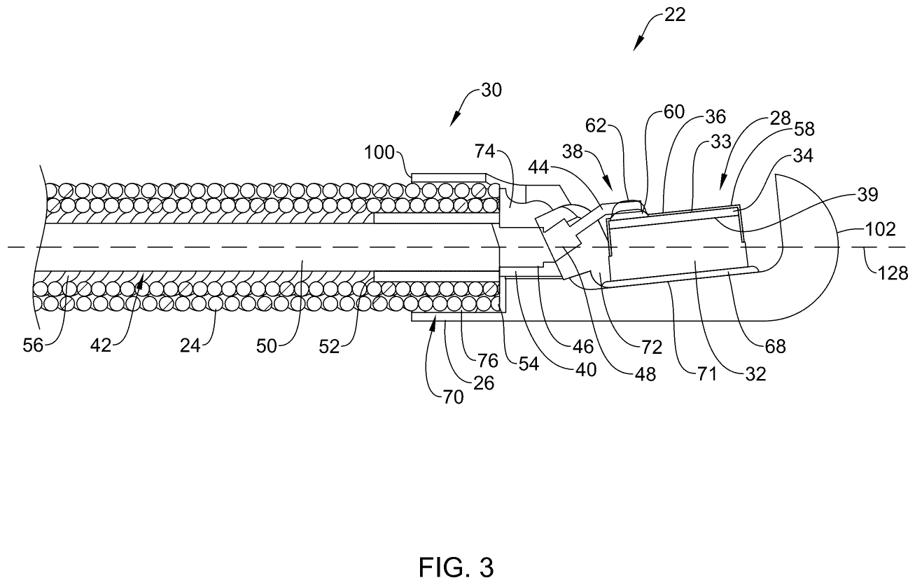

FIG. 2 is a side view of a distal end region 30 of the imaging assembly 22 and FIG. 3 is a cross-sectional view of the distal end region 30 of the imaging assembly 22. The distal end region 30 of the imaging assembly 22 may be configured to allow for an automated pick-and-place machine to assemble the transducer 28 into the housing 26. Further, electrical connections may be made by automatic dispensing of an electrically conductive adhesive (ECA) or solder, such as but not limited to a lead-free solder. Additionally, the present structure may allow for the electrical cable to be automatically laser stripped. For example, the electrical cable may be automatically laser or mechanically stripped prior to assembly or after assembly with the housing 26. While the present structure increases the manufacturability of the distal end region 30 of the imaging assembly 22, the performance of the imaging assembly 22 is not compromised. During use, the transducer 28 rotates at speeds of 1800 revolutions per minute (rpm), for example, and advances and/or retracts through a tortuous path. The housing 26 may protect the transducer 28 during use. Positioning the transducer 28 and housing 26 within the shaft 12 may protect the vessel from the spinning transducer 28 and drive cable 24. However, the housing 26 and/or transducer 28 cannot perforate the shaft 12. Further, minimal drag and/or friction should occur between the imaging assembly 22 and the shaft 12 to avoid non-uniform rotational distortion (NURD), non-uniform pullback distortion (NUPD), and mechanical failures.

The transducer 28 may include a backing layer 32, a layer with piezoelectric properties 34, such as, but not limited to, a lead zirconate titanate (PZT) layer with a layered metallized finish, and a matching layer 36. Some illustrative transducer configurations are described herein. Additional transducer configurations and methods of making transducers are described in commonly assigned patent application attorney docket number 2001.3748100 titled “Ultrasound Transducer For IVUS Catheters” filed of even date herewith, the disclosure of which is hereby incorporated by reference. The matching layer 36 may extend across or over an upper surface 33 with layered metallized finish and lateral and/or axial (e.g., proximal and distal) sides of the piezoelectric layer 34 as well as at least a portion of the lateral and/or axial sides of the backing layer 32. However, this is not required. In some embodiments, the matching layer 36 may extend across or over the upper surface 33 of the piezoelectric layer 34. The transducer 28 may be electrically coupled to an imaging system (not explicitly shown) through a first electrical connection 38 and a second electrical connection 40 via a third electrical connection 71. The first electrical connection 38 may electrically couple an upper surface 33 of the piezoelectric layer 34 with a central conductor 44 of a coaxial cable 42 when the matching layer 36 is locally removed. In some embodiments, the first electrical connection 38 may be a positive electrical connection. The second electrical connection 40 may electrically couple a lower surface or underside 39 of the piezoelectric layer 34 through backing material 32, the securement member 68, and the third electrical connection 71 with an annular conductive member 46 of a coaxial cable 42. In some embodiments, the second electrical connection 40 and the third electrical connection 71 may be negative electrical connections.

In some examples, the coaxial cable 42 may be an American Wire Gauge (AWG) coaxial cable. The coaxial cable 42 may have a characteristic impedance of in the range of about 50-66 Ohms to minimize reflections on the transmission line at both the load (e.g., transducer 28) and the source (e.g., transformer at the motor drive unit). The proximal end of the coaxial cable 42 may be electrically coupled to the transformer (not explicitly shown) of the imaging system.

The annular conductive member 46 may be electrically isolated from the central conductor 44 by an annular dielectric layer 48 and an outer insulative jacket 50 may be positioned around an outer surface of the annular conductive member 46. In some embodiments, the central conductor 44 may be a generally solid wire. For example, the central conductor 44 may be a silver-plated copper wire having a diameter in the range of about 0.0016 inches (45.72 micrometers (μm)). The diameter may be less than 0.0016 inches or greater than 0.0016 inches, as desired. Further, the central conductor 44 may be formed from other electrically conductive materials. The annular dielectric layer 48 may be formed from perfluoroalkoxy (PFA) or other dielectric materials that may be laser ablated. In some cases, doping the dielectric material with pigments that absorb the wavelength of the laser may help with ablation. In some examples, the annular dielectric layer 48 may have an outer diameter of about 0.0049 inches (121.9 μm). However, the outer diameter may be less than 0.0049 inches or greater than 0.0049 inches, as desired. The dielectric layer 48, along with the dimensions of the conductors 44, 46, may help maintain the approximately 50 Ohm characteristic impedance of the cable.

The annular conductive member 46 may be a braided, helically wound, or twisted member. In some cases, one or more filaments may be braided, wound, woven, or twisted to form the annular conductive member 46. The one or more filaments may be silver-plated copper. However, other electrically conductive materials may be used. The annular conductive member 46 may have an outer diameter of about 0.0065 inches (165.1 μm). However, the outer diameter may be less than 0.0065 inches or greater than 0.0065 inches, as desired. The outer insulative jacket 50 may be formed from an insulative material, such as, but not limited to, a polyester tape, and may be one or multiple layers of the same or opposite wrap directions. However, other insulative materials may be used, as desired. The outer insulative jacket 50 may have an outer diameter of about 0.0078 inches (198.1 μm). However, the outer diameter may be less than 0.0078 inches or greater than 0.0078 inches, as desired.

In addition to electrically coupling the lower surface 39 of the piezoelectric layer 34 with the imaging system, the annular conductive member 46 may have high coverage to reduce susceptibility to external sources or electrical noise. For example, the annular conductive member 46 may act as a second shield to block external electromagnetic interference (EMI). This may help maintain signal integrity. Further, the structure of the annular conductive member 46 may increase flexibility in the coaxial cable 42.

To assemble the coaxial cable 42 and the drive cable 24, the coaxial cable 42 is inserted into and through the lumen 56 of the drive cable 24. The coaxial cable 42 may be coupled with the inner surface of the drive cable 24 using, for example, an adhesive 52. Portions of the coaxial cable 42 may extend distally beyond a distal end 54 of the drive cable 24. After fixing the coaxial cable 42 to the drive cable 24, the coaxial cable 42 may be laser stripped and cut to precise lengths. For example, the central conductor 44 may extend distally beyond the annular dielectric layer 48. The annular dielectric layer 48 may extend distally beyond the annular conductive member 46. It is contemplated that the laser cut lengths of the central conductor 44, annular dielectric layer 48, and/or annular conductive member 46 may help control placement of the coaxial cable 42. For example, the laser cut lengths may help control placement of the annular conductive member 46 and the central conductor 44 relative to their respective attachment points. In some embodiments, the coaxial cable 42 may be formed into a desired shape prior to inserting the drive cable 24 and coaxial cable 42 assembly into the housing 26. For example, the distal end region of the coaxial cable 42 may be bent or curved to align the central conductor 44 with an upper surface 58 of the transducer 28. In some embodiments, the central conductor 44 may be bent or curved to align with a conductive member or connection point 60 which extends through or aligns with an upper surface of the matching layer 36 to electrically couple the central conductor 44 to the upper surface 33 of the piezoelectric layer 34. In some cases, the central conductor 44 may be soldered 62 to the conductive member 60. The conductive member 60 may be a solder material or “pre-tin solder” which was applied to the piezoelectric layer 34 during manufacture of the transducer 28. It is contemplated that soldering the central conductor 44 to a pre-tinned conductive member 60 may reduce or eliminate the need for cleaning the area of connection prior to securing the central conductor 44 to the conductive member 60. For example, applying pre-tin solder may make laser ablating easier. The pre-tin solder may act as a sacrificial layer. Without it, the laser may over-ablate the sputtered metal layer of the transducer 28, which may hinder the formation of an electrical connection to that same spot. However, in some cases, a region of the matching layer 36 may be removed to form the connection point and the central conductor 44 may be secured directly to the upper surface 33 of the piezoelectric layer 34. In yet another example, the central conductor 44 may be secured to the matching layer 36 directly, or to a pre-tinned solder that was exposed after removing and/or grinding the matching layer 36.

The second electrical connection 40 may include a solder or electrically conductive adhesive 74. The second electrical connection 40 may electrically couple the annular conductive member 46 with the housing 26 which in turn is connected to the bottom side 39 of the piezoelectric layer via the securement member 68 and the backing layer 32. A non-conductive adhesive 72 may be disposed over or encapsulate a distal end of the annular dielectric layer 48 of the coaxial cable. The non-conductive adhesive 72 may secure the central conductor 44 relative to the annular dielectric layer 48 such that proximal and/or distal movement of the central conductor 44 relative to the annular dielectric layer 48 is limited or prevented. Shifting of the central conductor 44 may cause the first electrical connection 38 to break.

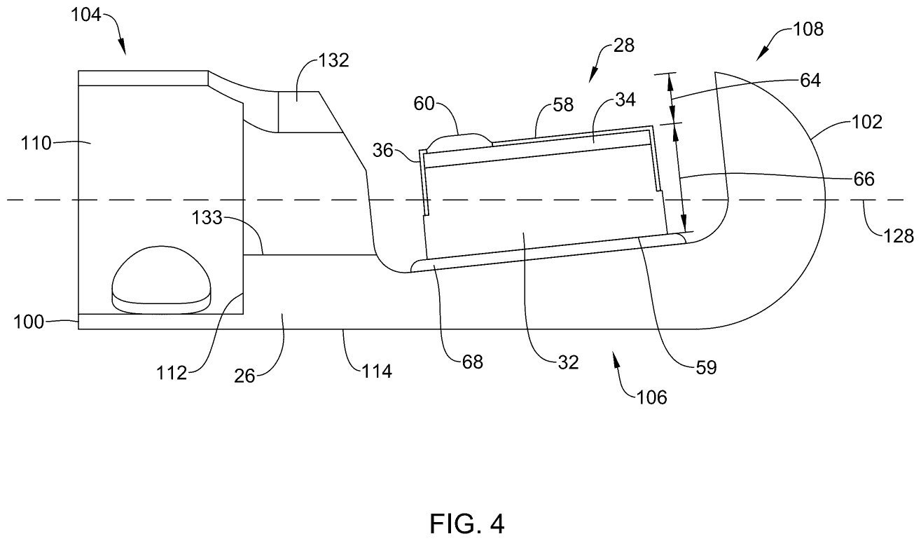

Referring additionally to FIG. 4, which is a cross-sectional view of the housing 26 and transducer 28, the transducer 28 may be sized and shaped to minimize a distance between the upper surface 58 of the transducer 28 and the inner surface of the shaft 12 (not explicitly shown in FIG. 4). However, the transducer 28 may be housed fully within the outer dimensions of the housing 26 such that the transducer 28 does not catch on, snag, or otherwise contact the shaft 12. In some embodiments, the distance 64 between a radially outward most portion, or the active portion, of the transducer 28 and the outer diameter of the housing 26 may be approximately 0.0043 inches (109.2 micrometers (μm)). However, distances of less than 0.0043 inches or greater than 0.0043 inches are also contemplated. Larger gaps between the surface of the transducer 28 and the inner surface of the shaft 12 may cause a reflection between the shaft 12 and transducer 28 to reverberate for a longer period of time, thus extending further into the image and obscuring the image data. Said differently, a shorter distance 64 between the upper surface 58 of the transducer 28 and the inner surface of the shaft 12 may reduce the time between echoes.

The transducer 28 may have a thickness 66 of approximately 0.0100 inches (254 μm) extending from the upper surface 58 to a lower surface 59 thereof. It is contemplated that the thickness of the transducer 28 may depend, at least in part, on the backing layer 32. The backing layer 32 may be formed from a highly attenuating material with a maximized thickness to maximize the attenuation of the backing layer 32. In some embodiments, the backing layer 32 may be formed from an epoxy. In other embodiments, the backing layer 32 may be formed from a tungsten and/or silver doped epoxy. Illustrative backing layers 32 are described in commonly assigned patent application attorney docket number 2001.3750100 titled “Backing Layer for Intravascular Imaging Device” filed of even date herewith, the disclosure of which is hereby incorporated by reference. A thicker backing layer 32 may provide more material to absorb and attenuate ultrasound waves that pass through the transducer 28. This increased attenuation may reduce internal reflections that can cause imaging artifacts. Further, an increased thickness may result in a longer time-of-flight for any residual reflections, which can help separate these reflections from the desired image data in the time domain.

Additionally, or alternatively, the housing 26 may include features configured to reduce reflection. For example, the difference in acoustic impedance at the interface behind the backing layer 32 (e.g., interface between the backing layer 32, securement member 68, and/or the housing 26) may be minimized. In one illustrative embodiment, some or all of the housing 26 may be formed from a polymer or a composite material (e.g., glass filled or metal filled) which may produce a lower impedance mismatch and thus a smaller reflection. It is contemplated that forming all or a portion of the housing 26 from a polymer may require the use of a thin metal plate or an extra component to form the negative electrical connection between the transducer and a coaxial cable. While metal may have a higher impedance mismatch, reducing the thickness to a small fraction of a wavelength will have minimal reflections and thus the mismatch between the backing layer 32 and the housing 26 will have more impact on the amplitude of the reflection.

In another example, reversing the phase of half of the energy reflected can allow for some phase cancellation. This can be done by having approximately one half of the interface area step up in acoustic impedance, and one half of the interface area step down in acoustic impedance per the reflection coefficient formula shown in Equation 1:

$$r = \frac{{z}_{L} - {z}_{O}}{{z}_{L} + {z}_{O}} \qquad Equation 1$$

where r is the amplitude reflection coefficient, Z_{L} is the impedance of the first material, and Z_{O} is the impedance of the second material. In the present example, half the surface area of the securement member 68 may be solder or electrically conductive adhesive (ECA) and half the surface area of the securement member 68 may be air bubbles. The optimal ratio of the surface area of the securement member 68 may be determined by ratio of the respective reflection coefficients.

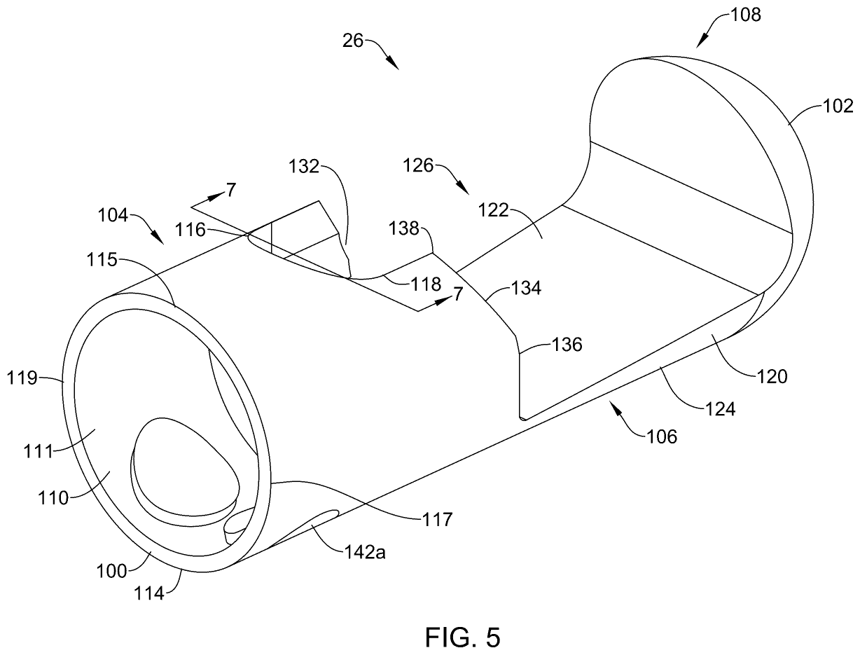

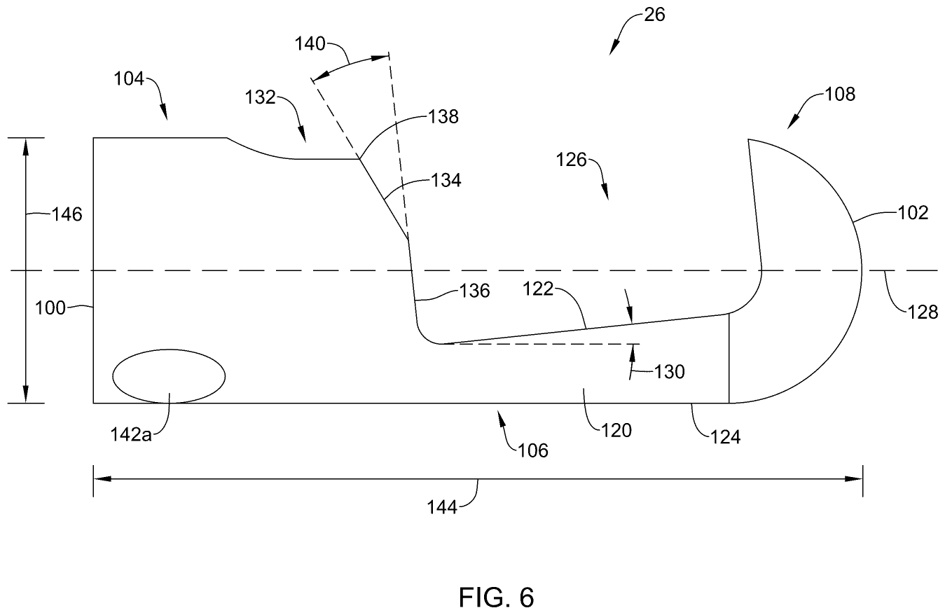

Referring additionally to FIG. 5 which illustrates a perspective view of the housing 26 and FIG. 6 which illustrates a side view of the illustrative housing 26, the housing 26 may have a generally cylindrical outer profile extending from a proximal end 100 to a distal end 102. To facilitate discussion of the housing 26, when viewing the housing 26 from the proximal end 100, a topside 115 of housing 26 generally corresponds to about 12 o'clock, a bottom side of the housing generally corresponds to 6 o'clock (first circumferential location 114), a first lateral side 117 generally corresponds to about 3 o'clock, and a second lateral side 119 generally corresponds to about 9 o'clock. The housing 26 may be formed from gold plated stainless steel. However, the housing 26 may be formed from other materials that provide strength to the distal end region 30 of the imaging assembly 22 and electrical conductivity for the second electrical connection 40. It is contemplated that the housing 26 may be machined, metal injection molded, 3D printed, or the like.

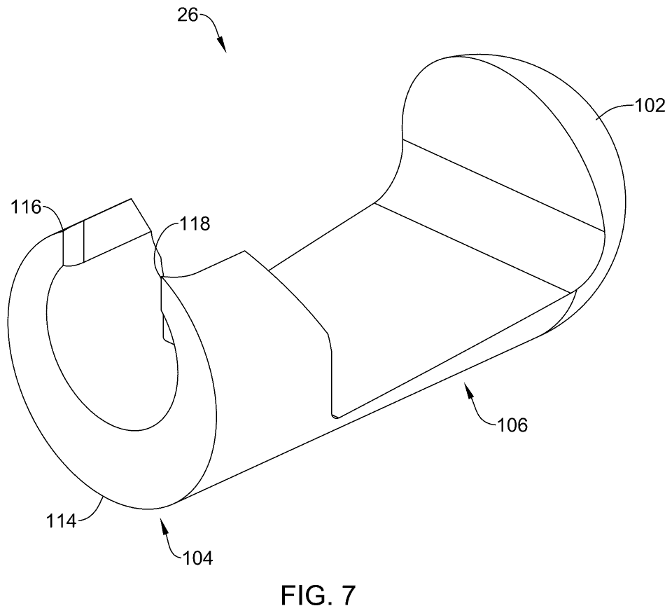

Generally, the housing 26 may transition between a generally tubular proximal end region 104, a generally open intermediate region 106, and a substantially solid distal end region 108. The transducer 28 and the electrical connections 38, 40 (not explicitly shown in FIGS. 4-6) may be disposed within the housing 26. The generally cylindrical outer profile of the housing 26 may help minimize drag and friction against the inner surface of the shaft 12, reducing the risk of NURD and NUPD. However, the housing 26 may take other shapes, as desired. The proximal end region 104 of the housing 26 may be generally tubular defining an opening 110 configured to receive a distal end region 70 (see, for example, FIG. 3) of the drive cable 24 therein. In some cases, the opening 110 may be generally circular. However, this is not required. The opening 110 may have a first diameter adjacent to the proximal end 100 and transition to a second, smaller diameter a distance from the proximal end 100 at a transition region 112 (FIG. 4). A wall thickness of the housing 26 distal to the transition region 112 may be greater than a wall thickness of the housing proximal to the transition region 112. In some embodiments, the wall thickness of the housing 26 distal to the transition region 112 may not be uniform. This is more clearly illustrated in FIG. 7 which is a cross-sectional view of the housing 26, taken at line 7-7 of FIG. 5. For example, the wall thickness of the housing 26 distal to the transition region 112 at a bottom or first circumferential location 114 may be thicker than a wall thickness at a second circumferential location 116 and a third circumferential location 118 each circumferentially spaced from the first circumferential location 114. For example, the wall thickness may gradually thin or reduce in thickness moving in both the clockwise and counterclockwise directions.

Returning to FIGS. 5 and 6, the transition region 112 may be an abrupt, stepwise transition in diameters. However, this is not required. The transition region 112 may create a mechanical stop to limit distal advancement of the drive cable 24 into the housing 26. The distal end region 108 of the housing 26 may have a substantially solid cross-section. In some cases, the distal end 102 of the housing 26 may have a rounded or domed configuration configured to limit or minimize damage to the inner surface of the shaft 12 and/or to the vessel.

An intermediate region 106 of the housing 26 may be disposed between the proximal end region 104 and the distal end region 108. The intermediate region 106 may include a substantially solid base 120 having a generally planar inner surface 122 and a curved outer surface 124. The intermediate region 106 may further include a cavity or recess 126 for receiving the transducer 28 and associated electrical connections. The cavity 126 may be laterally accessible from a direction generally orthogonal to or non-parallel to a longitudinal axis 128 (FIG. 2) of the imaging assembly 22. The curved outer surface 124 may extend 45° or less, 90° or less, 180° or less, etc. around the perimeter of the housing 26 relative to proximal end region 104. The generally planar inner surface 122 may extend at a non-parallel angle 130 relative to the longitudinal axis 128 of the imaging assembly 22. In some cases, the non-parallel angle 130 may be in the range of about 2° to about 8° or about 4° to about 6°. However, other angles 130 less than 2° or greater than 8° are also contemplated. However, due to the difference in speed of sound in water (approximately 1.5 millimeters per microsecond (mm/μs)) and the shaft material (approximately 2.6 mm/μs), large angles (e.g., approximately equal to or greater than 35°) may cause a total internal reflection and not enough energy may penetrate the shaft 12. The transducer 28 may be positioned on the planar inner surface 122 such that the surface of the transducer 28 extends generally parallel to the surface of the planar inner surface 122. This angled configuration may help minimize near-field artifacts caused by reflections between the transducer 28 and the shaft 12 relative to a transducer 28 that has been positioned with a surface extending generally parallel to the longitudinal axis 128 (e.g., parallel to the shaft 12).

The proximal end region 104 may include a recess, slot, or cut-out region 132. The cut-out region 132 may be generally circumferentially in line with an upper surface of the transducer 28 to facilitate electrical connections. The cut-out region may extend less than 45° about a circumference of the proximal end region 104. An inner cavity 111 (FIG. 5) of the proximal end region 104 may transition from a generally cylindrical lumen to a generally “U” shaped channel at or near a proximal end of the cut-out region 132. The proximal end of the cut-out region 132 may be axially aligned with or axially near the transition region 112. The interior portion of a wall 133 (see, for example, FIG. 4) opposing the cut-out region 132 may extend generally parallel to the longitudinal axis of the imaging assembly 22. This may allow the coaxial cable 42 to extend through the proximal end region 104 of the housing 26, or portions thereof, at an angle generally parallel to the longitudinal axis of the imaging assembly 22.

In some embodiments, the proximal end region 104 may include a chamfered or angled surface 134 extending between a distal end surface 136 of the proximal end region 104 and a distal end 138 of the cut-out region 132. The angled surface 134 may be positioned proximal to the intermediate region 106. The distal end surface 136 of the proximal end region 104 may extend generally orthogonal to the longitudinal axis 128 or about 90°±10°. In some cases, the angled surface 134 may extend at an angle 140 of approximately 25° relative to the distal end surface 136 of the proximal end region 104. However, the angle 140 may be less than 25° or greater than 25°, as desired. It is contemplated that the angled surface 134 may reduce retro-reflections by preventing or eliminating right angles near the transducer 28. For example, right angles (relative to the emitting surface of the transducer 28) near the transducer 28 can act as retroreflectors sending reflections back in the same direction where they came from which may contribute to near field artifacts (e.g., reflections between the transducer 28 and the shaft 12).

In some embodiments, the housing 26 may be mechanically and electrically coupled to the drive cable 24 by a welded joint, such as, but not limited to a laser-welded joint. The housing 26 may include one or more apertures 142a, 142b extending through a wall of the housing 26 adjacent the proximal end 100 thereof. The apertures 142a-b may be weld points for welding 76 (see, for example, FIG. 3) the housing 26 to the drive cable 24. In some cases, the housing 26 may include weld points near the first circumferential location 114 (e.g., on the bottom side or side opposite the cut-out region 132). However, this is not required. In some cases, the housing 26 may include pairs of weld points on opposing sides of the housing 26. For example, the housing 26 may include two weld points near a topside 115 (proximal to and adjacent to the cut-out region 132) and two weld points near the bottom side 114. In another example, the housing 26 may include two weld points near a first lateral side 117 and two weld points near a second opposing lateral side, or the like). The apertures 142a-b may be circumferentially offset from the identified circumferential reference points 114, 115, 117, 119. In another example, the housing 26 may include two or more weld points uniformly spaced along the circumference of the housing 26. For example, when two weld points are provided, the weld points may be spaced by about 180° anywhere around the circumference of the housing 26. In yet another example, the housing 26 may include a seam weld extending around an entire circumference (or portions thereof) of the housing 26. While the apertures 142a-b are illustrated as generally circular, the apertures 142a-b may take other shapes or forms. For example, the apertures 142a-b may be rectangular, square, elliptical, polygonal, or the like. Further the apertures 142a-b may be one or more slots or notches that extend distally from the proximal end 100 of the housing 26. It is further contemplated that welding may occur with no physical features on the housing 26. In yet other embodiments, the housing 26 may be free from apertures 142a-b, slots, notches, or the like. For example, the housing 26 and the drive cable 24 may be tack welded and the melt pool may through the wall of the housing 26 and into the drive cable 24, bonding the two components together.

The housing 26 may have a length 144 in the range of about 0.068 inches (1.723 millimeters). However, the length 144 may be greater than 0.068 inches or less than 0.068 inches. While increasing a length 144 of the housing 26 may increase ease of manufacturing, it is contemplated that shorter lengths may allow the system 10 to traverse more tortuous anatomy with greater ease and/or fewer failures. The housing 26 may have a maximum diameter 146 of about 0.024 inches (0.610 millimeters). However, the maximum diameter 146 may be greater than 0.024 inches or less than 0.024 inches.

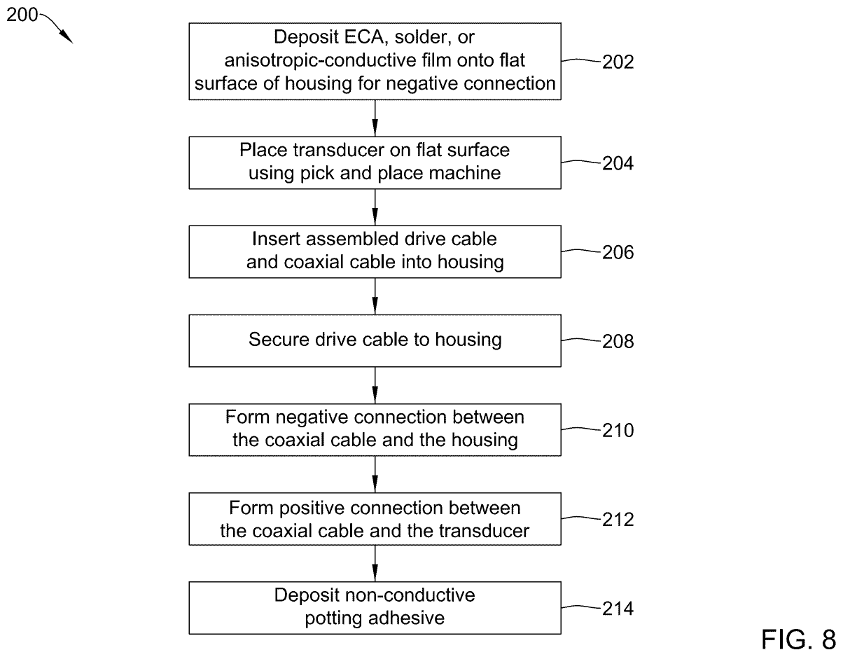

FIG. 8 is an illustrative flow chart 200 of a method for assembling the imaging assembly 22. While certain steps are shown or described as a sequence, in other embodiments fewer steps are contemplated and the order by which steps are performed can be different than what is illustrated. First, an electrically conductive adhesive (ECA), solder, or an anisotropic-conductive film may be deposited on the planar inner surface 122 of the housing 26 to form the securement member 68, as shown at block 202. The securement member 68 is electrically conductive to electrically couple the bottom side 39 of the piezoelectric layer 34 with the annular conductive member 46. In some embodiments, spherical beads may be added to the ECA to control the bond gap (e.g., a thickness of the securement member 68).

Next, the transducer 28 may be placed on the securement member 68 using an automated pick and place machine, as shown at block 204. However, this is not required. The transducer 28 may be placed by an operator or assembler, if so desired. A distal end region 70 of the drive cable 24 and the coaxial cable 42 may be inserted into proximal end region 104 of the housing 26, as shown at block 206. In some embodiments, the coaxial cable 42 may be secured to the drive cable 24 and prepared for connection prior to assembly with the housing 26. However, this is not required. In other embodiments, the coaxial cable 42 may be secured to the drive cable 24 after assembly with the housing 26. During insertion of the drive cable 24 and the coaxial cable 42 into the housing 26, the central conductor 44 may be forced upwards by the geometry of the housing 26 such that the central conductor 44 is positioned on or at the electrical connection 60 of the transducer 28. Alternatively, or additionally, the central conductor 44 may be shaped or formed so that the central conductor 44 sits directly above the electrical connection 60, as will be described in more detail herein. In some cases, the central conductor 44 may be manipulated by an operator or an automated machine to place the central conductor 44 at the electrical connection 60. The coaxial cable 42 may be laser stripped or mechanically stripped to remove a distal portion of the jacket 50, annular conductive member 46, and dielectric layer 48 to expose a distal end of the central conductor 44.

The drive cable 24 may be secured to the housing 26, as shown at block 208. In some cases, the drive cable 24 may be secured with an ECA or a non-conductive adhesive or epoxy. In other embodiments, the drive cable 24 may be secured with a weld. The apertures 142a, 142b in the housing 26 may provide a coupling or securement location, although the apertures 142a, 142b are not required. It is contemplated that welding the drive cable 24 to the housing 26 may form both a mechanical and an electrical connection between the drive cable 24 and the housing 26 (and ultimately the negative side 39 of the PZT layer 34.

The negative connection between the annular conductive member 46 of the coaxial cable 42 and the housing 26 may be made after the coaxial cable 42 is disposed within the housing 26, as shown at block 210. The slot or cut-out region 132 in the housing 26 may allow solder or ECA to be applied between the annular conductive member 46 and the housing 26. Said differently, the slot or cut-out region 132 creates an access point to dispense solder or ECA 74 into a lumen of proximal end region 104 of the housing 26 such that the solder or ECA 74 forms an electrical connection between the annular conductive member 46 and the housing 26. This may create an electrical path from the bottom side 39 of the piezoelectric layer 34 to the backing layer 32, through the securement member 68 to the housing 26, and through the solder or ECA 74 to the annular conductive member 46 and ultimately the transformer. Alternatively, the ECA may be applied to the annular conductive member 46 prior to mating the housing 26 with the drive cable 24.

Next, solder or ECA 62 may be deposited over the central conductor 44 and the electrical connection 60 to form a positive connection between the central conductor 44 and the transducer 28, as shown at block 212. The positive electrical path may flow from an upper surface 33 of the piezoelectric layer 34, through the connection point 60 and solder or ECA 62 to the central conductor 44 and to the transformer. Alternatively, an anisotropic conductive film may be used in place of ECA or solder. For example, an anisotropic conductive film may be placed on the transducer 28 prior to assembly into the housing, placed on a component already attached to the central conductor 44, or placed on the central conductor 44 prior to assembly.

Finally, a non-conductive potting adhesive 72 may be dispensed into the housing 26, as shown at block 214. The non-conductive potting adhesive 72 may be dispensed distal to the solder or ECA 74. The non-conductive potting adhesive 72 may secure the central conductor 44 relative to the dielectric layer 48 to prevent axial movement of the central conductor 44 relative to the dielectric layer 48 (e.g., in a direction generally parallel to the longitudinal axis 128 of the imaging assembly 22. Further, the non-conductive potting adhesive 72 may additionally secure the coaxial cable 42 to the housing 26.

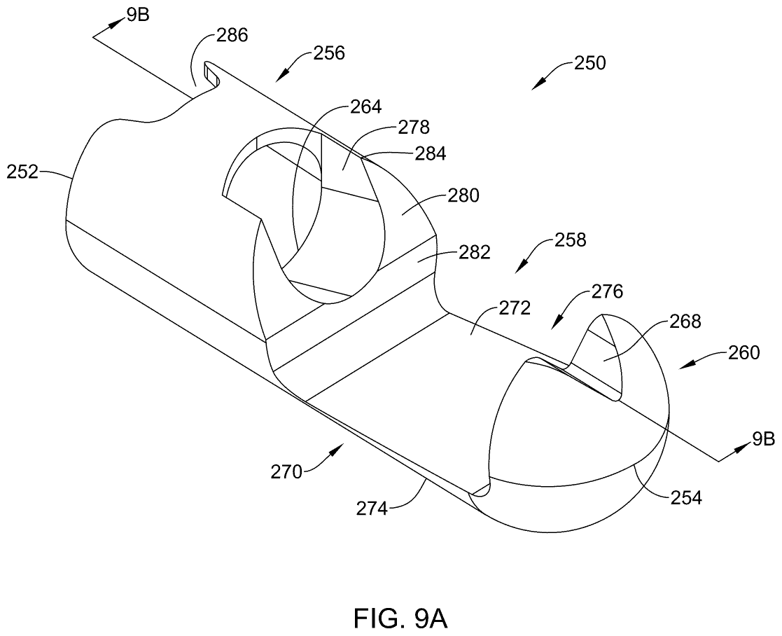

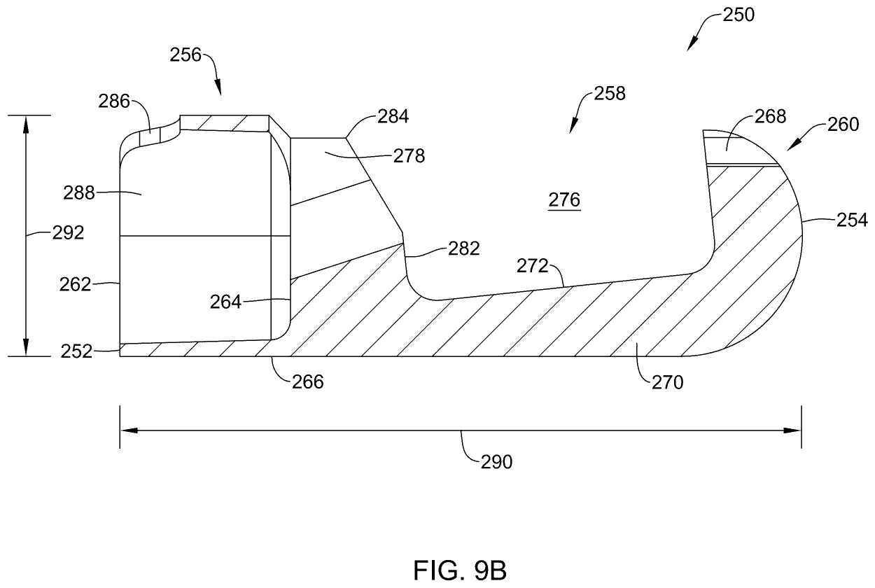

FIG. 9A is a perspective view of an alternative housing 250 and FIG. 9B is a cross-sectional view of the housing 250, taken at line 9B-9B of FIG. 9A. The housing 250 may be configured for use with an imaging assembly, such as imaging assembly 22 and a transducer, such as transducer 28. The housing 250 may have a generally cylindrical outer profile extending from a proximal end 252 to a distal end 254. The housing 250 may be formed from gold plated stainless steel. However, the housing 250 may be formed from other materials that provide strength to the distal end region 30 of the imaging assembly 22 and electrical conductivity for a negative electrical connection (e.g., similar in form and function to the second electrical connection 40 described herein). It is contemplated that the housing 250 may be machined, metal injection molded, 3D printed, or the like.

Generally, the housing 250 may transition between a generally tubular proximal end region 256, a generally open intermediate region 258, and a substantially solid distal end region 260. A transducer and the electrical connections (not explicitly shown in FIGS. 9A-9B) may be disposed within the housing 250. The generally cylindrical outer profile of the housing 250 may help minimize drag and friction against the inner surface of the shaft 12, reducing the risk of NURD and NUPD. However, the housing 250 may take other shapes, as desired. The proximal end region 256 of the housing 250 may be generally tubular defining an opening 262 configured to receive a distal end region of the drive cable 24 therein. In some cases, the opening 262 may be generally circular. However, this is not required. The opening 262 may have a first diameter adjacent to the proximal end 252 and transition to a second, smaller diameter a distance from the proximal end 252 at a transition region 264. A wall thickness of the housing 250 distal to the transition region 264 may be greater than a wall thickness of the housing proximal to the transition region 264. In some embodiments, the wall thickness of the housing 250 distal to the transition region 264 may not be uniform, similar to the wall thickness of the housing 26 shown and described with respect to FIG. 7. For example, the wall thickness of the housing 250 distal to the transition region 264 may be thickest at a bottom or first circumferential location 266 and may gradually thin or reduce in thickness moving in both the clockwise and counterclockwise directions.

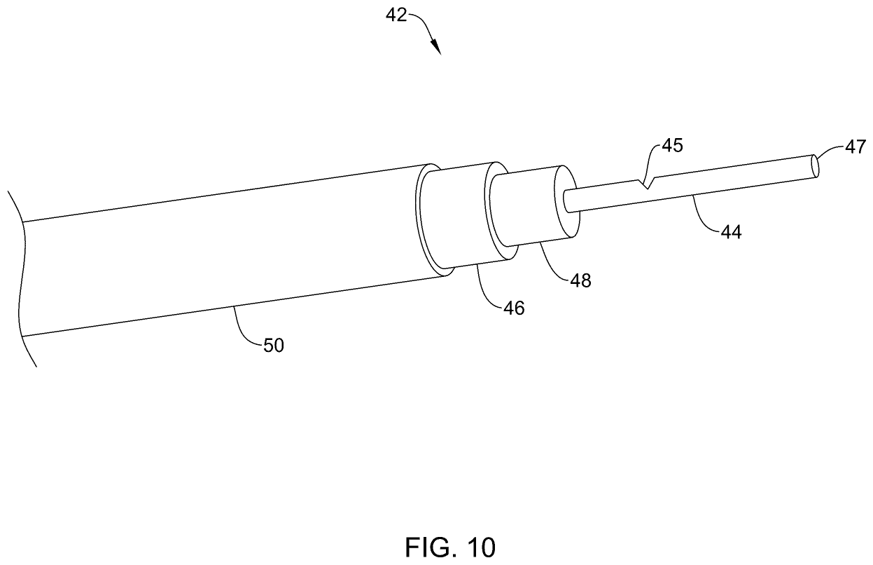

The transition region 264 may be an abrupt, stepwise transition in diameters. However, this is not required. The transition region 264 may create a mechanical stop to limit distal advancement of the drive cable 24 into the housing 250. The distal end region 260 of the housing 250 may have a substantially solid cross-section. In some cases, the distal end 254 of the housing 250 may have a rounded or domed configuration configured to limit or minimize damage to the inner surface of the shaft 12 and/or to the vessel. The distal end region 260 of the housing 250 may further include a notch or cut-out region 268. Generally, the notch 268 may be spaced approximately 180° from the first circumferential location 266. The notch 268 may be configured to receive the central conductor 44 therein. For example, as the coaxial cable 42 is being assembled with the housing 250, the central conductor 44 may be pulled through the notch 268. The notch 268 may be circumferentially aligned with the electrical connection 60 of the transducer 28 such that as the central conductor 44 is pulled through the notch 268, the central conductor 44 is aligned with the electrical connection 60 of the transducer 28. Referring additionally to FIG. 10, which illustrates a perspective schematic view of an illustrative coaxial cable 42, it is contemplated that the central conductor 44 may include a notch or cut out region 45 configured to create a failure point. For example, the central conductor 44 may be partially cut or notched during laser stripping to create the notch 45. The notch 45 may be located proximal to the distal end 47 of the central conductor 44 and distal to the electrical connection 60. After the central conductor 44 is connected or attached to the electrical connection 60, the central conductor 44 may be broken off at the notch 45 to remove the excess length (e.g., the length between the notch 45 and the distal end 47). It is contemplated that a coaxial cable 42 in which the central conductor 44 includes a notch 45 and is configured to extend distally beyond the electrical connection 60 during initial assembly may be used with any of the housing structures described herein. For example, a housing need not include the guiding notch 268 to be used with a central conductor 44 having a length in which the distal end 47 extends distally beyond the electrical connection 60 and is subsequently broken to remove the excess length.

Returning to FIGS. 9A and 9B, an intermediate region 258 of the housing 250 may be disposed between the proximal end region 256 and the distal end region 260. The intermediate region 258 may include a substantially solid base 270 having a generally planar inner surface 272 and a curved outer surface 274. The intermediate region 258 may further include a cavity or recess 276 for receiving the transducer 28 and associated electrical connections. The cavity 276 may be laterally accessible from a direction generally orthogonal to or non-parallel to a longitudinal axis of the imaging assembly 22. The curved outer surface 274 may extend 45° or less, 90° or less, 180° or less, etc. around the perimeter of the housing 250 relative to proximal end region 256. The generally planar inner surface 272 may extend at a non-parallel angle relative to the longitudinal axis of the imaging assembly 22. In some cases, the non-parallel angle may be in the range of about 2° to about 8° or about 4° to about 6°. However, other angles less than 2° or greater than 8° are also contemplated. However, due to the difference in speed of sound in water (approximately 1.5 millimeters per microsecond (mm/μs)) and the shaft material (approximately 2.6 mm/μs), large angles (e.g., approximately equal to or greater than 35°) may cause a total internal reflection and not enough energy may penetrate the shaft 12. The transducer 28 may be positioned on the planar inner surface 272 such that the surface of the transducer 28 extends generally parallel to the surface of the planar inner surface 272. This angled configuration may help minimize near-field artifacts caused by reflections between the transducer 28 and the shaft 12 relative to a transducer 28 that has been positioned with a surface extending generally parallel to the longitudinal axis (e.g., parallel to the shaft 12).

The proximal end region 256 may include a recess or slot 278 extending through a sidewall of the housing 250. The slot 278 may be generally circumferentially in line with an upper surface of the transducer 28 to facilitate electrical connections. The slot 278 may extend less than 45° about a circumference of the proximal end region 256. An inner cavity 288 of the proximal end region 256 may transition from a generally cylindrical lumen to a generally “U” shaped channel at a proximal end of the slot 278. The proximal end of the slot 278 may be axially aligned with or axially near the transition region 264.

In some embodiments, the proximal end region 256 may include a chamfered or angled surface 280 extending between a distal end surface 282 of the proximal end region 256 and a distal end 284 of the slot 278. The angled surface 280 may be positioned proximal to the intermediate region 258. The distal end surface 282 of the proximal end region 256 may extend generally orthogonal to the longitudinal axis or about 90°±10°. In some cases, the angled surface 280 may extend at an angle of approximately 25° relative to the distal end surface 282 of the proximal end region 256. However, the angle may be less than 25° or greater than 25°, as desired. It is contemplated that the angled surface 280 may reduce retro-reflections by preventing or eliminating right angles near the transducer 28. For example, right angles (relative to the emitting surface of the transducer 28) near the transducer 28 can act as retroreflectors sending reflections back in the same direction where they came from which may contribute to near field artifacts (e.g., reflections between the transducer 28 and the shaft 12).

In some embodiments, the housing 250 maybe mechanically and electrically coupled to the drive cable 24 by a welded joint, such as, but not limited to a laser-welded joint. The housing 250 may include a recess 286 extending through a wall of the housing 250 adjacent the proximal end 252 thereof. The recess 286 may be a weld point for welding the housing 250 to the drive cable 24. In the illustrated embodiment, the recess 286 extends distally from the proximal end 252 of the housing 250. However, this is not required. The recess 286 may be distally spaced from the proximal end 252, if so desired. In the illustrated embodiment, the recess 286 is generally circumferentially aligned with the slot 278. However, this is not required. When a single recess 286 is provided, the recess 286 may be positioned anywhere about the circumference of the housing 250, as desired. The housing 250 may include any number of recesses 286 desired. Further, when more than one recess 286 is provided, the recesses 286 may be evenly or eccentrically spaced around a circumference of the housing 250, as desired. In other embodiments, the housing 250 may include apertures similar in form and function to the apertures 142a-b described herein. In yet another example, the housing 250 may include a seam weld extending around an entire circumference (or portions thereof) of the housing 250. In yet other examples, the housing 250 may be free from apertures, slots, notches, or the like.

The housing 250 may have a length 290 in the range of about 0.068 inches (1.723 millimeters). However, the length 290 may be greater than 0.068 inches or less than 0.068 inches. While increasing a length 290 of the housing 250 may increase ease of manufacturing, it is contemplated that shorter lengths may allow the system 10 to traverse more tortuous anatomy with greater ease and/or fewer failures. The housing 250 may have a maximum diameter 292 of about 0.024 inches (0.610 millimeters). However, the maximum diameter 292 may be greater than 0.024 inches or less than 0.024 inches.

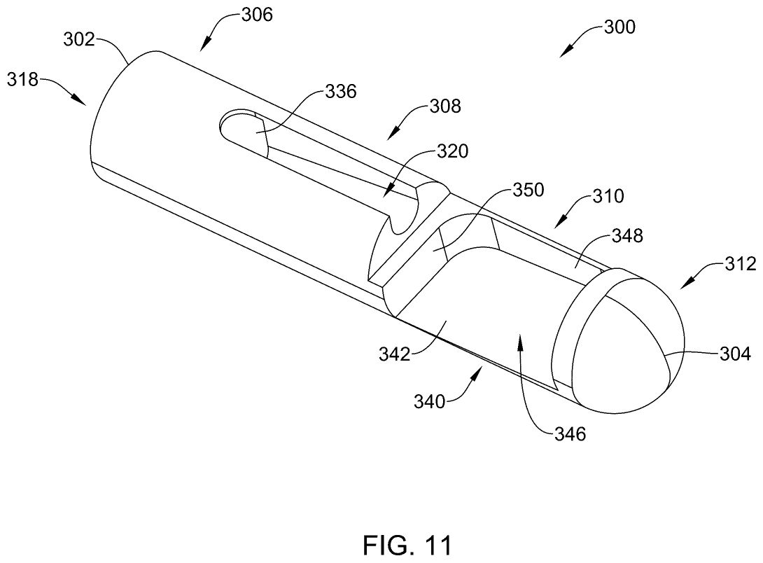

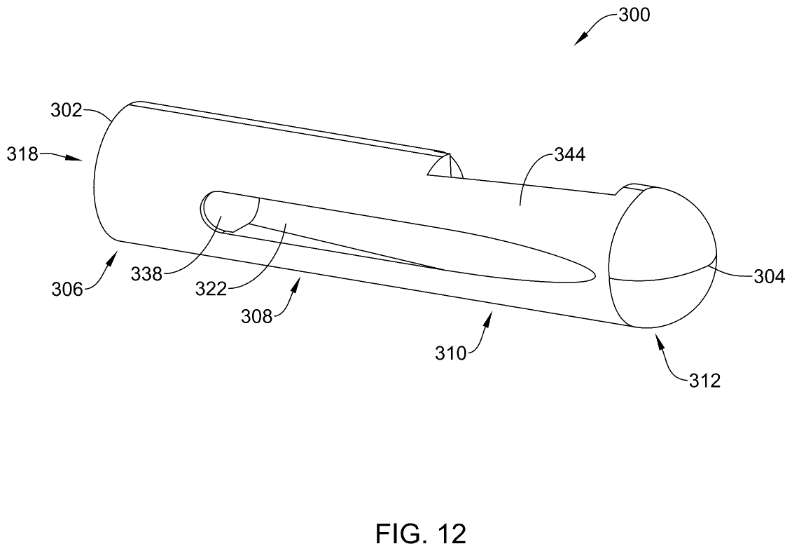

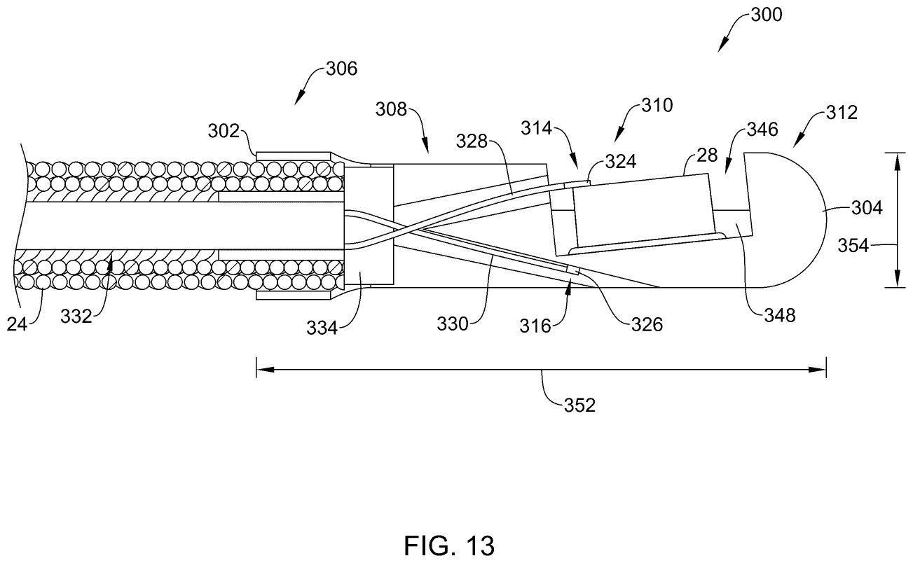

FIG. 11 is a perspective view of an alternative housing 300. FIG. 12 is a perspective view of the illustrative housing 300 of FIG. 11 rotated clockwise by about 90° relative to FIG. 11. FIG. 13 is a partial cross-sectional view of the illustrative housing 300 with a drive cable 24, electrical conductors 324, 326, and transducer 28. The housing 300 may be configured for use with an imaging assembly, such as imaging assembly 22 and a transducer, such as transducer 28. The housing 300 may have a generally cylindrical outer profile extending from a proximal end 302 to a distal end 304. The housing 300 may be formed from gold plated stainless steel. However, the housing 300 may be formed from other materials that provide strength to the distal end region 30 of the imaging assembly 22 and electrical conductivity for a negative electrical connection 316 (e.g., similar in form and function to the second electrical connection 40 described herein). It is contemplated that the housing 300 may be machined, metal injection molded, 3D printed, or the like.

Generally, the housing 300 may transition between a generally tubular proximal end region 306, a first channeled intermediate region 308, a second generally open intermediate region 310, and a substantially solid distal end region 312. A transducer 28 and the electrical connections 314, 316 may be disposed within the housing 300. The generally cylindrical outer profile of the housing 300 may help minimize drag and friction against the inner surface of the shaft 12, reducing the risk of NURD and NUPD. However, the housing 300 may take other shapes, as desired. The proximal end region 306 of the housing 300 may be generally tubular defining an opening 318 configured to receive a distal end region of the drive cable 24 therein. In some cases, the opening 318 may be generally circular. However, this is not required.

The first channeled intermediate region 308 may be substantially solid with a first channel 320 and a second channel 322 each extending radially inwards from an outer perimeter of the housing 300 and extending along a length thereof. The first channel 320 may be accessed from a lumen 334 of the proximal end region 306 via a first opening 336. Similarly, the second channel 322 may be accessed from the lumen 334 of the proximal end region via a second opening 338. Aside from the openings 336, 338, the channels 320, 322 may be bounded by a generally U-shaped wall. The depths of the channels 320, 322 may vary along the length thereof. For example, the depths of the channels 320, 322 may decrease in the distal direction. It is contemplated that channels 320, 322 may extend distally from a distal end of the proximal end region 306.

The first and second channels 320, 322 may be spaced approximately 180° from one another. For example, the first channel 320 may be positioned to align a positive electrical conductor 324 with an upper surface of the transducer 28 and the second channel 322 may be positioned to align a negative electrical conductor 326 with a portion of the housing 300 adjacent to the bottom of the transducer 28. The first and second channels 320 may have a depth such that the electrical conductors 324, 326 are fully disposed within the perimeter of the housing 300. Said differently, the first and second channels 320 may have a depth such that the electrical conductors 324, 326 do not extend radially beyond the outer perimeter of the housing 300. The electrical conductors 324, 326 may replace the central conductor 44 and the annular conductive member 46 of the coaxial cable 42 described herein. Each of the electrical conductors 324, 326 may include a dielectric coating 328, 330 covering the outer surface thereof. The dielectric coating 328, 330 may be removed from the electrical conductors 324, 326 at distal ends thereof or at a location adjacent to the electrical connections 314, 316. The electrical conductors 324, 326 may be secured to the transducer 28 or the housing 300, respectively, with an ECA or solder, as desired. It is contemplated that the channels 320, 322 may guide the electrical conductors 324, 326 to the desired coupling or securement location. In some examples, the negative electrical conductor 326 may be shorter than the positive electrical conductors 324 without impacting the signal path length to the transducer 28. The electrical conductors 324, 326 may extend through a tubular member 332 such as a wrap, braided member, or the like.

The distal end region 312 of the housing 300 may have a substantially solid cross-section. In some cases, the distal end 304 of the housing 300 may have a rounded or domed configuration configured to limit or minimize damage to the inner surface of the shaft 12 and/or to the vessel.

The second intermediate region 310 of the housing 300 may be disposed between the first intermediate region 308 and the distal end region 312. The intermediate region 310 may include a substantially solid base 340 having a generally planar inner surface 342 and a curved outer surface 344. The intermediate region 310 may further include a cavity or recess 346 for receiving the transducer 28 and associated electrical connections. The cavity 346 may be laterally accessible from a direction generally orthogonal to or non-parallel to a longitudinal axis of the imaging assembly 22. The curved outer surface 344 may extend 45° or less, 90° or less, 180° or less, etc. around the perimeter of the housing 300 relative to proximal end region 306. The generally planar inner surface 342 may extend at a non-parallel angle relative to the longitudinal axis of the imaging assembly 22. In some cases, the non-parallel angle may be in the range of about 2° to about 8° or about 4° to about 6°. However, other angles less than 2° or greater than 8° are also contemplated. However, due to the difference in speed of sound in water (approximately 1.5 millimeters per microsecond (mm/μs)) and the shaft material (approximately 2.6 mm/μs), large angles (e.g., approximately equal to or greater than 35°) may cause a total internal reflection and not enough energy may penetrate the shaft 12. The transducer 28 may be positioned on the planar inner surface 342 such that the surface of the transducer 28 extends generally parallel to the surface of the planar inner surface 342. This angled configuration may help minimize near-field artifacts caused by reflections between the transducer 28 and the shaft 12 relative to a transducer 28 that has been positioned with a surface extending generally parallel to the longitudinal axis (e.g., parallel to the shaft 12).

The second intermediate region 310 may include a first wall 348 extending generally orthogonal to the generally planar surface 342 and parallel to a longitudinal axis of the housing 300. The first wall 348 may be configured to extend generally parallel to a lateral side of the transducer 28 to retain potting compound (not explicitly shown in FIG. 13) and to protect the advancing-side of the transducer 28 during rotation. The second intermediate region 310 may include a second wall 350 extending generally orthogonal to the generally planar surface 342 and orthogonal to a longitudinal axis of the housing 300. In some configurations, the second intermediately region 310 may be free from either or both of the walls 348, 350.

In some embodiments, the housing 300 maybe mechanically and electrically coupled to the drive cable 24 by a welded joint, such as, but not limited to a laser-welded joint. While not explicitly shown, the housing 300 may include one or more recesses, one or more apertures, or the like which may function as a weld point for welding the housing 300 to the drive cable 24. However, recesses or apertures are not required. In another example, the housing 300 may include a seam weld extending around an entire circumference (or portions thereof) of the housing 300.

The housing 300 may have a length 352 in the range of about 0.068 inches (1.723 millimeters). However, the length 352 may be greater than 0.068 inches or less than 0.068 inches. While increasing a length 352 of the housing 300 may increase ease of manufacturing, it is contemplated that shorter lengths may allow the system 10 to traverse more tortuous anatomy with greater ease and/or fewer failures. The housing 300 may have a maximum diameter 354 of about 0.024 inches (0.610 millimeters). However, the maximum diameter 354 may be greater than 0.024 inches or less than 0.024 inches.

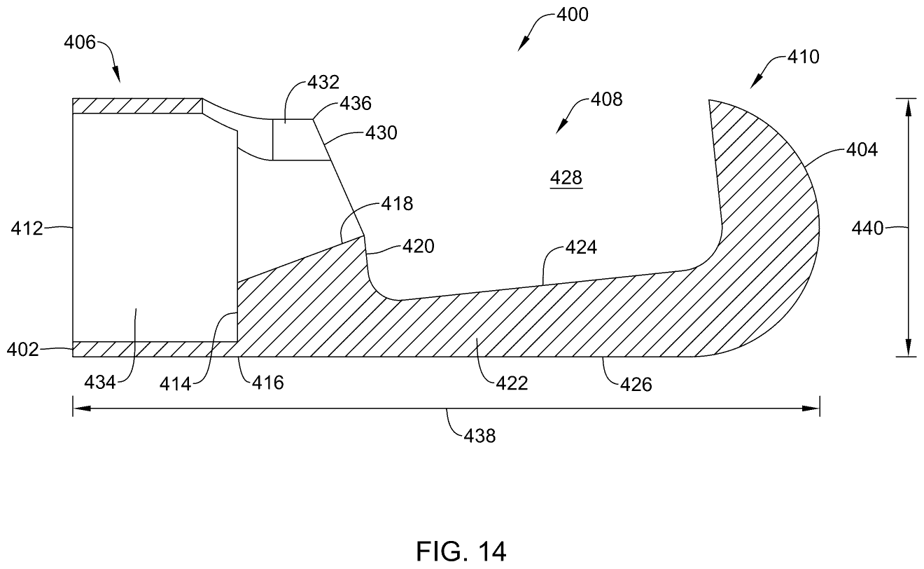

FIG. 14 is a cross-sectional view of another illustrative housing 400. The housing 400 may be configured for use with an imaging assembly, such as imaging assembly 22 and a transducer, such as transducer 28. The housing 400 may have a generally cylindrical outer profile extending from a proximal end 402 to a distal end 404. The housing 400 may be formed from gold plated stainless steel. However, the housing 400 may be formed from other materials that provide strength to the distal end region 30 of the imaging assembly 22 and electrical conductivity for a negative electrical connection (e.g., similar in form and function to the second electrical connection 40 described herein). It is contemplated that the housing 400 may be machined, metal injection molded, 3D printed, or the like.

Generally, the housing 400 may transition between a generally tubular proximal end region 406, a generally open intermediate region 408, and a substantially solid distal end region 410. The transducer 28 and the electrical connections 38, 40 (not explicitly shown in FIG. 14) may be disposed within the housing 400. The generally cylindrical outer profile of the housing 400 may help minimize drag and friction against the inner surface of the shaft 12, reducing the risk of NURD and NUPD. However, the housing 400 may take other shapes, as desired. The proximal end region 406 of the housing 400 may be generally tubular defining an opening 412 configured to receive a distal end region of the drive cable 24 therein. In some cases, the opening 412 may be generally circular. However, this is not required. The opening 412 may have a first diameter adjacent to the proximal end 402 and transition to a second, smaller diameter a distance from the proximal end 402 at a transition region 414. As will be described in more detail herein, a portion of the wall of the housing 400 may be removed distal to the transition region 414. In such an instance, the second diameter may refer to a maximum dimension of a generally “U” shaped channel. A wall thickness of the housing 400 distal to the transition region 414 may be greater than a wall thickness of the housing proximal to the transition region 414. In some embodiments, the wall thickness of the housing 400 distal to the transition region 414 may not be uniform in the circumferential direction, similar to the wall thickness of the housing 26 shown and described with respect to FIG. 7. For example, the wall thickness of the housing 400 distal to the transition region 414 may be thickest at a bottom or first circumferential location 416 and may gradually thin or reduce in thickness moving in both the clockwise and counterclockwise directions. Further, the wall thickness at the first circumferential location 416 may increase in thickness in the distal direction. This may create an angled surface 418 extending from the transition region 414 to a distal end 420 of the proximal end region 406. The angled surface 418 may extend at a non-parallel angle relative to a longitudinal axis of the housing 400. The angled surface 418 may direct the coaxial cable 42 upwards towards an upper surface of the transducer 28.

The transition region 414 may be an abrupt, stepwise transition in diameters. However, this is not required. The transition region 414 may create a mechanical stop to limit distal advancement of the drive cable 24 into the housing 400. The distal end region 410 of the housing 400 may have a substantially solid cross-section. In some cases, the distal end 404 of the housing 400 may have a rounded or domed configuration configured to limit or minimize damage to the inner surface of the shaft 12 and/or to the vessel.

An intermediate region 408 of the housing 400 may be disposed between the proximal end region 406 and the distal end region 410. The intermediate region 408 may include a substantially solid base 422 having a generally planar inner surface 424 and a curved outer surface 426. The intermediate region 408 may further include a cavity or recess 428 for receiving the transducer 28 and associated electrical connections. The cavity 428 may be laterally accessible from a direction generally orthogonal to or non-parallel to a longitudinal axis of the imaging assembly 22. The curved outer surface 426 may extend 45° or less, 90° or less, 180° or less, etc. around the perimeter of the housing 400 relative to proximal end region 406. The generally planar inner surface 424 may extend at a non-parallel angle relative to the longitudinal axis of the imaging assembly 22. In some cases, the non-parallel angle may be in the range of about 2° to about 8° or about 4° to about 6°. However, other angles less than 2° or greater than 8° are also contemplated. However, due to the difference in speed of sound in water (approximately 1.5 millimeters per microsecond (mm/μs)) and the shaft material (approximately 2.6 mm/μs), large angles (e.g., approximately equal to or greater than 35°) may cause a total internal reflection and not enough energy may penetrate the shaft 12. The transducer 28 may be positioned on the planar inner surface 424 such that the surface of the transducer 28 extends generally parallel to the surface of the planar inner surface 424. This angled configuration may help minimize near-field artifacts caused by reflections between the transducer 28 and the shaft 12 relative to a transducer 28 that has been positioned with a surface extending generally parallel to the longitudinal axis (e.g., parallel to the shaft 12).

The proximal end region 406 may include a recess, slot, or cut-out region 432. The cut-out region 432 may be generally circumferentially in line with an upper surface of the transducer 28 to facilitate electrical connections. The cut-out region may extend less than 45° about a circumference of the proximal end region 406. An inner cavity 434 of the proximal end region 406 may transition from a generally cylindrical lumen to a generally “U” shaped channel at or near a proximal end of the cut-out region 432. The proximal end of the cut-out region 432 may be axially aligned with or axially near the transition region 414.

In some embodiments, the proximal end region 406 may include a chamfered or angled surface 430 extending between a distal end surface 420 of the proximal end region 406 and a distal end 436 of the cut-out region 432. The angled surface 430 may be positioned proximal to the intermediate region 408. The distal end surface 420 of the proximal end region 406 may extend generally orthogonal to the longitudinal axis or about 90°±10°. In some cases, the angled surface 430 may extend at an angle of approximately 25° relative to the distal end surface 420 of the proximal end region 406. However, the angle may be less than 25° or greater than 25°, as desired. It is contemplated that the angled surface 430 may reduce retro-reflections by preventing or eliminating right angles near the transducer 28. For example, right angles (relative to the emitting surface of the transducer 28) near the transducer 28 can act as retroreflectors sending reflections back in the same direction where they came from which may contribute to near field artifacts (e.g., reflections between the transducer 28 and the shaft 12).

In some embodiments, the housing 400 maybe mechanically and electrically coupled to the drive cable 24 by a welded joint, such as, but not limited to a laser-welded joint. While not explicitly shown, the housing 400 may include one or more recesses, one or more apertures, or the like which may function as a weld point for welding the housing 400 to the drive cable 24. However, the housing 400 may be free from recesses, apertures, or the like. In another example, the housing 400 may include a seam weld extending around an entire circumference (or portions thereof) of the housing 400.

The housing 400 may have a length 438 in the range of about 0.068 inches (1.723 millimeters). However, the length 438 may be greater than 0.068 inches or less than 0.068 inches. While increasing a length 438 of the housing 400 may increase ease or manufacturing, it is contemplated that shorter lengths may allow the system 10 to traverse more tortuous anatomy with greater ease and/or fewer failures. The housing 400 may have a maximum diameter 440 of about 0.024 inches (0.610 millimeters). However, the maximum diameter 440 may be greater than 0.024 inches or less than 0.024 inches.

A plurality of transducers 28 may be formed from a single block having a length and width greater than a length and width of the transducer 28. To begin, a composite PZT wafer or structure may be formed. However, in some embodiments, the PZT wafer may be a generally solid PZT wafer. Generally, a composite PZT wafer may be formed by mating two diced PZT wafers with an epoxy disposed therebetween. In another example, a thin layer of a polymer coating (i.e., parylene) may be added to the diced PZT surface prior to mating the diced PZT wafers to facilitate thermal bonding of the assembly. The PZT wafers may be heated and pressed together to bond the two wafers which may provide a reduced profile composite wafer which is easier to process.

Once the PZT wafer is constructed (composite or solid), the PZT wafer may be optionally mounted to tape or a glass plate. In some cases, prior to mounting the PZT wafer, an upper and lower surface of the PZT wafer may be sputter coated. Next, a backing layer (similar in form and function to backing layer 32) may be cast onto the PZT wafer. In some embodiments, the backing layer may be a conductive epoxy. The backing layer may be cast flat, cured, and then ground or lapped to a precise thickness. A coating consisting of sputtered, layered metals may be coated on the bottom surface (e.g., surface opposite the surface contacting the PZT wafer) of the backing layer. The metallic coating may enhance the electrical connection between the backing layer 32 and a securement member 68 (see, for example, FIG. 2). Further, the metallic coating may reduce or eliminate cross-linking between the backing layer 32 and the tape used to process the transducer block. In some examples, the securement member 68 may be a solder bond. It is contemplated that a thickness of the metallic layers may be increased to improve reliability of the coupling between the securement member 68 and the transducer 28. In some examples, the metallic coating may provide a low resistance electrical connection between individual transducers 28 during manufacture (e.g., before singulation) for electrical testing.

Next, a matching layer (similar in form and function to matching layer 36) may be applied to the surface of the PZT layer. In some embodiments, the matching layer may be applied to only the PZT layer. In other embodiments, the matching layer may extend along lateral and/or axial sides of the backing layer. In some embodiments, a conductive connective region may be applied or pre-tinned to the PZT layer prior to applying the matching layer. A region of the matching layer may be removed or ablated to provide a connection point 60 (see, for example FIG. 3) for a first electrical connection 38 (FIG. 3) connecting a positive lead to the upper metallized surface of the PZT layer. The connection point 60 may be generally circular or may take other shapes, such as, but not limited to square, rectangular, oblong, or the like. The connection point 60 may be either a metallized surface of the PZT wafer or the pre-tin solder.

Next, the PZT wafer and backing layer assembly may be flipped and the optional mount removed from the PZT wafer. The wafer and backing layer assembly or block may be mounted to a dicing tape. A dicing saw having a first blade thickness may be used to dice or cut the PZT wafer and backing layer assembly into an array of transducer elements including a plurality of rows and a plurality of columns. The dice or cut may extend partially through an entire thickness of the backing layer such that at least some regions of the array remain at least partially connected at the backing layer. In some embodiments, the rows of the array of transducer elements may be separated (e.g., the cut extends through an entire thickness of the PZT wafer/backing layer block) while the columns remain mechanically and electrically connected through the backing layer and sputter coating.

Next, the transducers may be electrically tested prior to singulation. The array may be diced such that that the transducers forming the columns are interconnected by a region of backing layer and metallic coating while the transducers forming the rows are spaced from one another by a gap. Said differently, the transducers forming the rows may be free from connection at the backing layer and/or metallic coating. However, this is not required. The transducers forming the rows may be connected. The reverse configuration is also contemplated in which the transducers forming the rows are interconnected and the transducers forming the columns are free from connection. Additional quality control testing is described in commonly assigned patent application attorney docket number 2001.3748100 titled “Ultrasound Transducer For IVUS Catheters” filed of even date herewith, the disclosure of which is hereby incorporated by reference.

In some embodiments, a ledge or step or region of exposed backing layer may be present on at least one transducer of the plurality of interconnected transducers. To perform the test, a negative lead may be coupled with or placed in contact with the backing layer at the ledge or step of a first column and a positive lead may be coupled to or placed in contact with the connection point of a first transducer. An electrical property, such as, but not limited to, impedance, may be measured to verify the electrical functioning of the transducer. Other electrical properties may be used as desired. To test the next transducer, the negative lead may remain coupled to the ledge or step as the backing layer and metallic coatings of the interconnected transducers are physically connected and electrically connected. The positive lead may be coupled to or placed in contact with the connection point of a second transducer. Again, an electrical property, such as, but not limited to, impedance, may be measured to verify the electrical functioning of the transducer. The positive lead may be repositioned to the connection point of each remaining transducer in the column, and the process repeated for each transducer in the column. When testing has been performed on each transducer of the first column, the negative lead may be coupled to the backing layer or step of the first transducer of the next column and the process repeated. This may continue for each column of the array. It is contemplated that minimizing the number of different connections to be completed may reduce the amount of time required to test a plurality of transducers.

After completion of the testing, the transducers may be singulated. Singulation may be performed with a dicing saw. In some cases, the dicing saw may have a blade thickness that is similar to the blade thickness used to dice the array into columns and rows. In other examples, the dicing saw may have a blade thickness that is thinner than the blade thickness used to dice the array into columns and rows such that the transducers include a region of backing layer having a first length greater than a second length adjacent to the metallized PZT layer. Said differently, the dicing saw may remove less backing layer material during singulation than during the first dicing step, which may create axially (and/or laterally) extending edges or feet. During singulation, the ledge or step used to perform the testing may be removed or the transducers including the ledge or step 228 may be disposed of. In some embodiments, the transducers may be singulated with a die-expansion method. After singulation, the transducers 28 may all be oriented the same, in a flat uniform array, fixed in place. This may make it easy to pick-and-place them manually or with an automated machine.

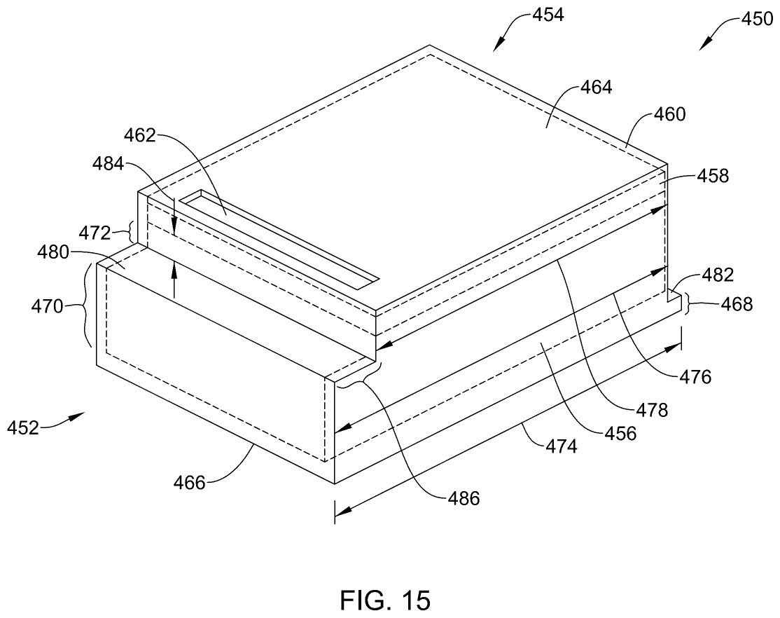

FIG. 15 is a perspective view of an illustrative transducer 450 extending from a proximal end 452 to a distal end 454. The transducer 450 includes a backing layer 456, a PZT layer 458, and a matching layer 460. A region of the matching layer 460 has been removed or ablated to define a connection point 462. In FIG. 15, the connection point 462 may be generally rectangular. It is contemplated that a generally rectangular or strip-like connection point 462 may allow an electrically conductive adhesive or solder to be dispensed in a line along the proximal edge of the active portion of the transducer 450 (e.g., proximal edge of the PZT layer 458) to couple the PZT layer 458 with a coaxial cable. The connection point 462 may be adjacent to a proximal end 452 of the transducer 450. The matching layer 460 may extend across an upper surface 464 of the PZT layer 458 and along the lateral and axial (proximal and distal) sides of the PZT layer 458 and the lateral and axial (proximal and distal) sides of an entirety of a perimeter the backing layer 456. The lower or bottom surface 466 of the backing layer 456 may remain free from the matching layer 460. However, in some embodiments, the matching layer 460 may be disposed over less than an entirety of the lateral and/or axial (proximal and distal) surfaces of the backing layer 456.

The transducer 450 may include a lower portion 468 adjacent to a bottom surface 466 thereof, an upper portion 472 adjacent to the PZT layer 458, and an intermediate portion 470 therebetween. The lower portion 468 of the backing layer 456 may have a first length 474. The intermediate portion 470 may have a second length 476 less than the first length 474. The upper portion 472 may have a third length 478 less than the second length 476. Generally, the length of the backing layer 456 may increase from an upper surface adjacent to the PZT layer 458 to the lower or bottom surface 466 in an incremental or stepwise manner to define a first ledge 480 and a second ledge or foot 482. The second ledge or foot 482 may extend distally beyond a distal end of the intermediate portion 470. Alternatively, or additionally, the second ledge or foot 482 may extend proximally beyond a proximal end of the intermediate portion 470. In yet another example, the transducer 450 may include a ledge (similar to ledge 482) extending both proximally and distally beyond the proximal and distal ends of the intermediate portion 470. The thickness of the ledge 482 may be determined during the initial dicing. The ledge 482 may provide the electrical connection between adjacent transducers during the manufacturing process (e.g., when the transducers 450 are in an array such as array). The ledge 482 may be created when a dicing saw is used for singulation which has a thinner blade than the dicing saw used for initial dicing. For example, less material may be removed during singulation with a thinner blade.

The first ledge 480 may also be manufactured during the initial dicing. For example, the initial dicing may include an additional dicing step in which the dicing blade is used to create a second cut extending in the lateral direction in which the second cut extends through less of the thickness of the PZT wafer/backing layer block than the first cut. This may remove a proximal portion of the PZT wafer and a thickness 484 and length 486 of the backing layer to define the first ledge 480. The first ledge 480 extends proximally from the proximal end of the PZT layer 458. The first ledge 480 may support a non-conductive potting adhesive which is used to secure the portions of a coaxial cable 42 to the transducer 450 and/or housing 26. It is contemplated that the ledge 480 may reduce the volume of potting material required to secure the coaxial cable. Alternatively, or additionally, the ledge 480 may support a ECA or solder for electrically coupling the connection point 462 with the central conductor 44. The matching layer 460 may extend over and cover the first ledge 480 to prevent electrical shorts between the first electrical connection 38 and the backing layer 456.

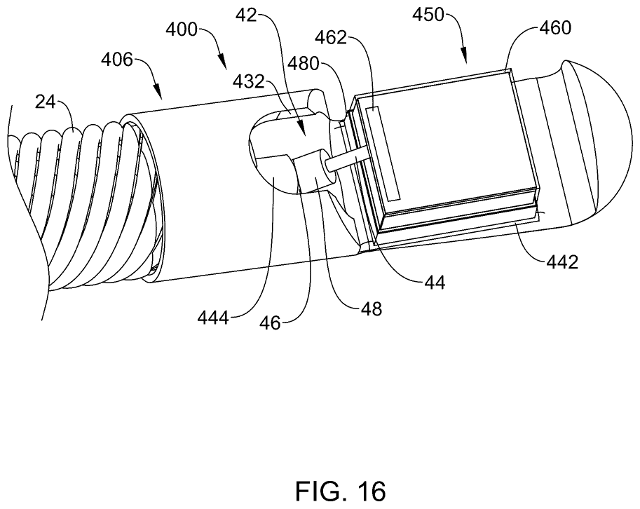

FIGS. 16-19 illustrate sequential steps of an illustrative method and mechanism for electrically coupling the transducer 450 of FIG. 15 with the housing 400 of FIG. 14. Generally, in FIGS. 16-19, the central conductor 44 may be positioned proximal to the connection point 462. Referring to FIG. 16, the transducer 450 has been secured to the housing 400 at a securement member 442. The securement member 442 may be solder, ECA, or the like. In some embodiments, a proximal end of the transducer 450 (e.g., at or near ledge 480) may be positioned against or abutting the housing 400 to facilitate placement of transducer 450. The coaxial cable 42 has been secured to the drive cable 24 and laser or mechanically stripped to expose the central conductor 44 and the annular conductive member 46. The drive cable 24 and coaxial cable 42 assembly are secured to the housing 400 (e.g., via welding, ECA, soldering, or like). As the drive cable 24 and coaxial cable 42 assembly are inserted into the housing 400, a distal end of the central conductor 44 may rest on the ledge 480 of the transducer 450. It is contemplated that the central conductor 44 may not require forming or manipulating to rest on the ledge 480 whereas forming or manipulating might be required to position the central conductor on an upper surface of the transducer 450 (such as, on the connection point 462). The annular conductive member 46 is electrically coupled with the housing with an electrical connection 444, such as, but not limited to solder or ECA. For example, the material may be dispensed through the cut-out region 432 to couple the annular conductive member 46 electrically and mechanically with the housing 400 to complete the negative connection between the backside of the piezoelectric layer 458 and the transformer.

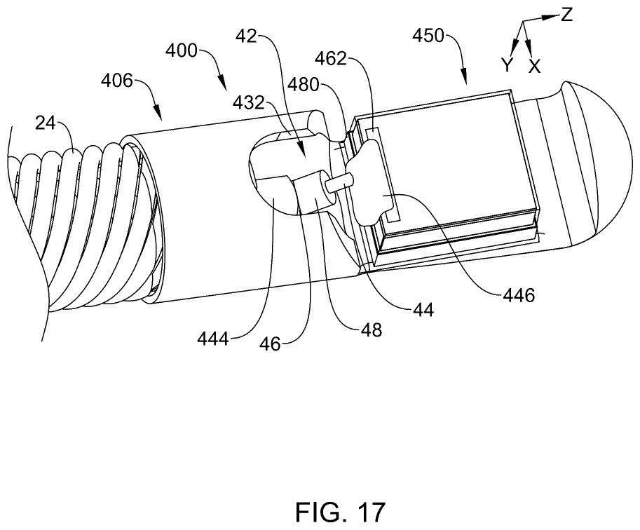

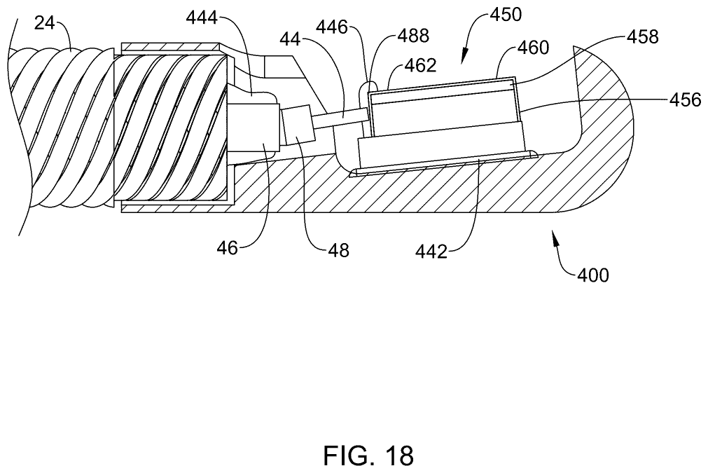

Referring to FIG. 17 and FIG. 18, which is a partial cross-sectional view of FIG. 17, the central conductor 44 may be electrically coupled to the connection point 462 (and thus the transducer 450) via a second electrical connection 446 to form the positive connection between the transformer and the transducer 450. The second electrical connection 446 may be an ECA that is dispensed in a line along the proximal edge 488 of the active portion of the transducer 450. The ECA may drip or run off the proximal edge 488 of the active portion of the transducer 450 and land or rest on the ledge 480. In addition to contacting the ledge 480, the ECA contacts the central conductor 44 to form the electrical connection 446 between the connection point 462 of the transducer 450 and the central conductor 44. As the ledge 480 is coated with the generally non-conductive matching layer 460, the excess ECA may rest on the ledge 480 without shorting to the negative side of the circuit. It is contemplated that dispensing the ECA in a line along the proximal edge 488 of the active portion of the transducer 450 may minimize the area of the ECA over the aperture of the transducer 450 (e.g., the active area that transits or receives acoustic waves). This may increase the sensitivity of the transducer 450 and reduce the beam width. Further, as the ECA is dispensed laterally along the proximal edge 488 of the active portion of the transducer 450 and runs down onto the ledge 480 along a same width, the ECA may contact the central conductor 44 with more accuracy. This may increase the tolerance in the Y and Z directions with the tolerance in the X direction controlled by the width of the connection point 462. While the second electrical connection 446 is described with respect to an ECA, other electrically conductive materials may be used as well, such as, but not limited to, solder.

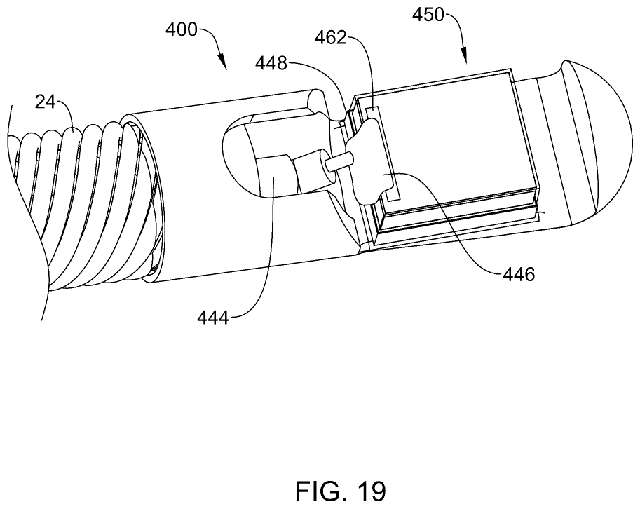

Referring now to FIG. 19, after the connection point 462 and the central conductor 44 are electrically coupled, a non-conductive potting adhesive 448 may be dispensed into the housing 400. The non-conductive potting adhesive 448 may be configured to mechanically secure the central conductor 44 to the annular conductive member 46 to prevent or reduce axial movement of the central conductor 44 (e.g., movement in a direction generally parallel to the longitudinal axis of the housing 400). It is contemplated that the ledge 480 may support some of the excess non-conductive potting adhesive 448 to limit spread of the non-conductive potting adhesive 448 outside of the housing 400. Further, the non-conductive potting adhesive 448 may fill in any gaps between a bottom surface of the transducer 450 and the housing 400.

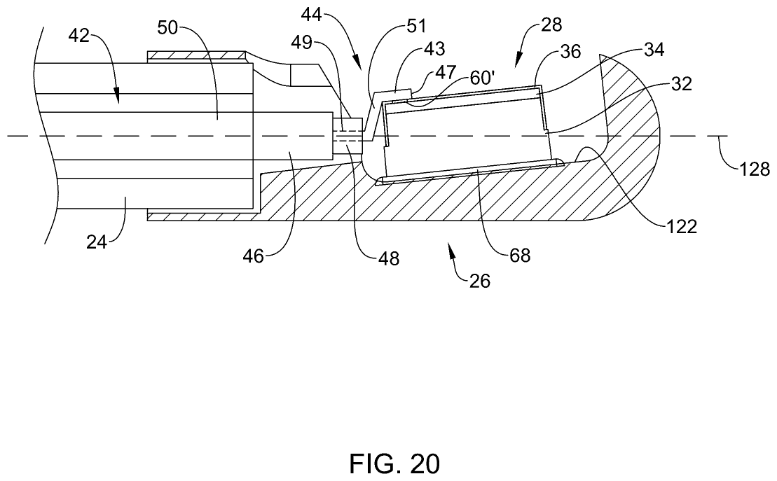

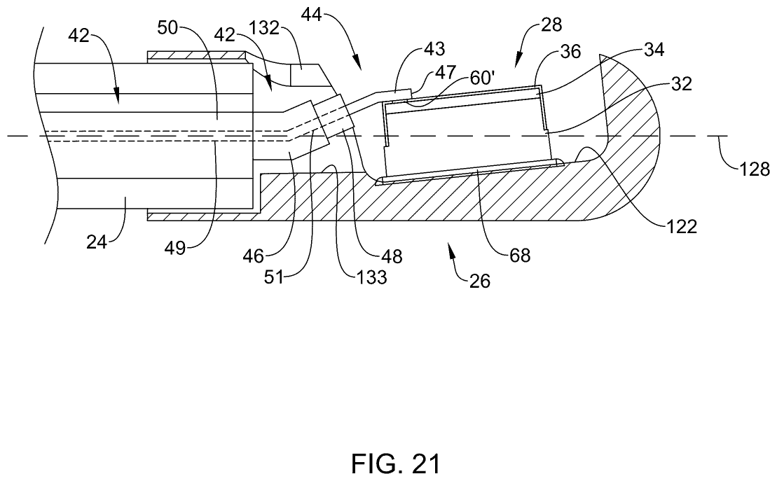

FIGS. 20-23 illustrate partial cross-sectional views of illustrative distal end regions of an imaging assembly which include alternative shapes and configurations of the coaxial cable 42. In FIGS. 20 and 21, the housing 26 may include a straight lead-in for the coaxial cable 42. Said differently, the interior portion of the wall 133 opposing the cut-out region 132 may extend generally parallel to the longitudinal axis of the imaging assembly 22. Referring to FIG. 20, a distal end region of the central conductor 44 may be curved or bent to position the distal end 47 of the central conductor 44 above the transducer 28. This may be performed prior to inserting the drive cable 24 and coaxial cable 42 assembly into the housing 26. In some examples, the central conductor 44 may be bent into the desired shape prior to assembling the coaxial cable 42 with the drive cable 24. However, this is not required. To bend the coaxial cable 42 into the desired shape, the jacket 50, annular conductive member 46, and dielectric layer 48 may be stripped or laser removed from the coaxial cable 42 from the distal end 47 to a location proximal to the transducer 28. The exposed portion of the central conductor 44 (e.g., free from the dielectric layer 48) may be placed into a die and bent into the desired configuration. The straight lead-in of the housing 26 may allow the pre-bent central conductor 44 to be inserted into the housing 26 without the distal end 47 of the central conductor 44 contacting the interior wall 133 of the housing 26. This may help prevent or limit unintentional deformation of the pre-bent shape.

A distalmost end region 43 of the central conductor 44 may extend generally parallel to the planar inner surface 122. A proximal region 49 of the central conductor 44 may extend generally parallel to the longitudinal axis 128 of the imaging assembly 22. The proximal region 49 and the distalmost end region 43 may be interconnected by an intermediate region 51. The intermediate region 51 may extend at a non-parallel angle relative to the planar inner surface 122 and/or the longitudinal axis 128 of the imaging assembly 22. In some embodiments, the intermediate region 51 may extend generally orthogonal to the longitudinal axis 128 of the imaging assembly 22. The intermediate region 51 may extend at other non-parallel angles relative to the longitudinal axis 128 of the imaging assembly 22, such as, but not limited to, in the range of about 75° to 105° relative to the longitudinal axis 128 of the imaging assembly 22. For example, the central conductor 44 may have a “Z” or “S” shape adjacent to the transducer 28. In some cases, the Z” or “S” shape may be stretched or elongated. It is contemplated that the positioning the intermediate region 51 generally orthogonal to the longitudinal axis 128 of the imaging assembly 22 may allow the annular conductive member 46 and the dielectric layer 48 to remain generally parallel to the longitudinal axis 128 of the imaging assembly 22 while still positioning the distalmost end region 43 adjacent to the connection point 60′ of the transducer 28. In FIG. 20, the connection point 60′ is a region where the matching layer 36 has been removed to expose the piezoelectric layer 34.

While FIG. 20 does not illustrate the electrical connections between the coaxial cable 42 and the transducer 28/housing 26, it should be understood that these connections are made to complete the electrical circuit. For example, the annular conductive member 46 may be electrically coupled to the housing via solder or ECA, as shown and described with respect to FIG. 3. Additionally, the central conductor 44 may be electrically coupled to the connection point 60′ with solder or ECA, as shown and described with respect to FIG. 3. After the electrical connections are made, a non-conductive potting adhesive (e.g., 72 in FIG. 3) may be dispensed over the exposed central conductor 44. The non-conductive potting adhesive may grip directly onto the exposed central conductor 44 and cover it. This may protect the central conductor 44 from tensile forces as well as abrasion. For example, the non-conductive potting adhesive may help prevent the central conductor 44 from shifting within the coaxial cable 42. For example, shifting of the central conductor 44 may create tension in the central conductor 44 which may put stress on the connection between the central conductor 44 and the transducer 28. Further, the Z” or “S” shape of the central conductor 44 may position the central conductor 44 such that most of the exposed central conductor 44 is covered and protected by the non-conductive potting adhesive. The central conductor 44 may protrude above the surface of the transducer 28 just proximal to the transducer 28 to make the electrical connection. Minimizing the exposed central conductor 44 may maximize the protection of the central conductor 44 and the connection between the central conductor 44 and the transducer 28. In some embodiments, the exposed portion of the central conductor 44 may include surface texturing or roughening to increase adhesion between the central conductor 44 and the non-conductive potting adhesive.

FIG. 21 illustrates an alternative bend configuration for the coaxial cable 42. A distal end region of the central conductor 44, the annular conductive member 46, and the dielectric layer 48 may be curved or bent to position the distal end 47 of the central conductor 44 above the transducer 28. This may be performed prior to inserting the drive cable 24 and coaxial cable 42 assembly into the housing 26. In some examples, the central conductor 44, the annular conductive member 46, and the dielectric layer 48 may be bent into the desired shape prior to assembling the coaxial cable 42 with the drive cable 24. However, this is not required. To bend the coaxial cable 42 into the desired shape, the dielectric layer 48 may be stripped or laser removed from the jacket 50, annular conductive member 46, and dielectric layer 48 from the distal end 47 to a location proximal to the transducer 28. The exposed portion of the central conductor 44 (e.g., free from the dielectric layer 48), a distal end region of the annular conductive member 46, and a distal end region of the dielectric layer 48 may be placed into a die and bent into the desired configuration. The straight lead-in of the housing 26 may allow the pre-bent coaxial cable 42 to be inserted into the housing 26 without the distal end 47 of the central conductor 44 contacting the interior wall 133 of the housing 26. This may help prevent or limit unintentional deformation of the pre-bent shape.

A distalmost end region 43 of the central conductor 44 may extend generally parallel to the planar inner surface 122. A proximal region 49 of the central conductor 44 may extend generally parallel to the longitudinal axis 128 of the imaging assembly 22. The proximal region 49 and the distalmost end region 43 may be interconnected by an intermediate region 51. The intermediate region 51 may extend at a non-parallel angle relative to the planar inner surface 122 and/or the longitudinal axis 128 of the imaging assembly 22. In some embodiments, the intermediate region 51 may extend at angle of about 45° to the longitudinal axis 128 of the imaging assembly 22. The intermediate region 51 may extend at other non-parallel angles relative to the longitudinal axis 128 of the imaging assembly 22, such as, but not limited to, in the range of about 30° to 70° relative to the longitudinal axis 128 of the imaging assembly 22. For example, the central conductor 44 may have a more gentle or gradual curve (relative to the abrupt transition of FIG. 20) adjacent to the transducer 28. It is contemplated that bending the annular conductive member 46 and the dielectric layer 48 may allow more of the central conductor 44 to remain covered by the dielectric layer 48 while still positioning the distalmost end region 43 adjacent to the connection point 60′ of the transducer 28. In FIG. 21, the connection point 60′ is a region where the matching layer 36 has been removed to expose the piezoelectric layer 34.

While FIG. 21 does not illustrate the electrical connections between the coaxial cable 42 and the transducer 28/housing 26, it should be understood that these connections are made to complete the electrical circuit. For example, the annular conductive member 46 may be electrically coupled to the housing via solder or ECA, as shown and described with respect to FIG. 3 (e.g., 74 in FIG. 3). Additionally, the central conductor 44 may be electrically coupled to the connection point 60′ with solder or ECA (e.g., 62 in FIG. 3), as shown and described with respect to FIG. 3. After the electrical connections are made, a non-conductive potting adhesive (e.g., 72 in FIG. 3) may be dispensed over the exposed central conductor 44 and the distal end region of the dielectric layer 48. In some cases, the non-conductive potting adhesive may be dispensed over the distal end region of the annular conductive member 46. The non-conductive potting adhesive may grip directly onto the exposed central conductor 44 and cover portions of it. This may protect the central conductor 44 from tensile forces as well as abrasion. For example, the non-conductive potting adhesive may help prevent the central conductor 44 from shifting within the coaxial cable 42. For example, shifting of the central conductor 44 may create tension in the central conductor 44 which may put stress on the connection between the central conductor 44 and the transducer 28. Further, the bent shape of the central conductor 44 may position the central conductor 44 such that most of the exposed central conductor 44 is covered and protected by the non-conductive potting adhesive. The central conductor 44 may protrude above the surface of the transducer 28 just proximal to the transducer 28 to make the electrical connection. Minimizing the exposed central conductor 44 may maximize the protection of the central conductor 44 and the connection between the central conductor 44 and the transducer 28. In some embodiments, the exposed portion of the central conductor 44 may include surface texturing or roughening to increase adhesion between the central conductor 44 and the non-conductive potting adhesive.

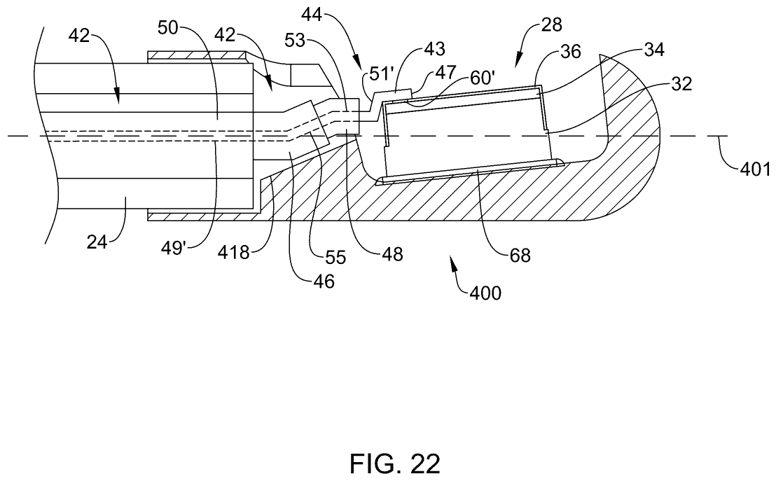

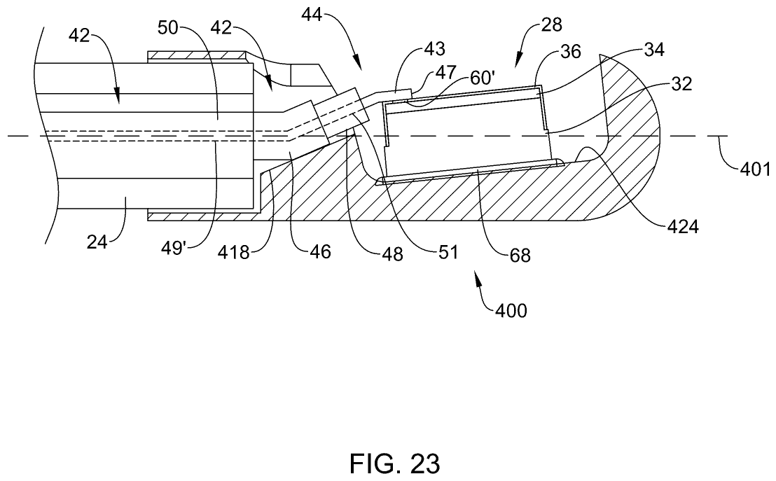

In FIGS. 22 and 23, the housing 400 may include an angled lead-in for the coaxial cable 42. Said differently, the angled surface 418 of an interior wall of the housing 400 opposite the cut-out region 432 may extend at a non-parallel angle relative to a longitudinal axis 401 of the housing 400. The angled surface 418 may direct the coaxial cable 42 upwards towards an upper surface of the transducer 28. Referring to FIG. 22, a distal end region of the coaxial cable 42 may be curved or bent to position the distal end 47 of the central conductor 44 above the transducer 28. This may be performed prior to inserting the drive cable 24 and coaxial cable 42 assembly into the housing 400. In some examples, the central conductor 44 may be bent into the desired shape prior to assembling the coaxial cable 42 with the drive cable 24. However, this is not required. To bend the coaxial cable 42 into the desired shape, the dielectric layer 48 may be stripped or laser removed from the jacket 50, annular conductive member 46, and dielectric layer 48 from the distal end 47 to a location proximal to the transducer 28. The exposed portion of the central conductor 44 (e.g., free from the dielectric layer 48), a distal end region of the annular conductive member 46, and a distal end region of the dielectric layer 48 may be placed into a die and bent into the desired configuration. The angled lead-in of the housing 400 may help guide the pre-bent central conductor 44 within the housing 400.

The coaxial cable 42 may include more than one bend or more than one transition. A distalmost end region 43 of the central conductor 44 may extend generally parallel to the planar inner surface 424. A proximal-most region 49′ of the central conductor 44 may extend generally parallel to the longitudinal axis 401 of the housing 400. A central intermediate region 53 may also extend generally parallel to the longitudinal axis 401 of the housing 400. The proximal-most region 49′ and the central intermediate region 53 may be interconnected by a second intermediate region 55. The second intermediate region 55 may extend at a non-parallel angle relative to the planar inner surface 424 and/or the longitudinal axis 401 of the housing 400. In some embodiments, the second intermediate region 55 may extend at angle of about 45° to the longitudinal axis 401 of the housing 400. The second intermediate region 55 may extend at other non-parallel angles relative to the longitudinal axis 401 of the housing 400, such as, but not limited to, in the range of about 30° to 70° relative to the longitudinal axis 401 of the housing 400. The distal end region of the annular conductive member 46 may extend generally parallel to the second intermediate region 55 of the central conductor 44.

The distalmost region 43 and the central intermediate region 53 may be interconnected by a third intermediate region 51′. The third intermediate region 51′ may extend at a non-parallel angle relative to the planar inner surface 424 and/or the longitudinal axis 401 of the housing 400. In some embodiments, the second intermediate region 55 may extend at angle of about 60° to the longitudinal axis 401 of the housing 400. The second intermediate region 55 may extend at other non-parallel angles relative to the longitudinal axis 401 of the housing 400, such as, but not limited to, in the range of about 45 to 85° relative to the longitudinal axis 401 of the housing 400. The distal end region of the dielectric layer 48 may extend generally parallel to the central intermediate region 23 of the central conductor 44. The plurality of bent segments may create more gradual transitions to position the distalmost end region 43 adjacent to the connection point 60′ of the transducer 28. In FIG. 22, the connection point 60′ is a region where the matching layer 36 has been removed to expose the piezoelectric layer 34.

While FIG. 22 does not illustrate the electrical connections between the coaxial cable 42 and the transducer 28/housing 400, it should be understood that these connections are made to complete the electrical circuit. For example, the annular conductive member 46 may be electrically coupled to the housing via solder or ECA, as shown and described with respect to FIG. 3. Additionally, the central conductor 44 may be electrically coupled to the connection point 60′ with solder or ECA, as shown and described with respect to FIG. 3. After the electrical connections are made, a non-conductive potting adhesive (e.g., 72 in FIG. 3) may be dispensed over the exposed central conductor 44. The non-conductive potting adhesive may grip directly onto the exposed central conductor 44 and cover it. This may protect the central conductor 44 from tensile forces as well as abrasion. For example, the non-conductive potting adhesive may help prevent the central conductor 44 from shifting within the coaxial cable 42. For example, shifting of the central conductor 44 may create tension in the central conductor 44 which may put stress on the connection between the central conductor 44 and the transducer 28. Further, the shape of the central conductor 44 may position the central conductor 44 such that most of the exposed central conductor 44 is covered and protected by the non-conductive potting adhesive. The central conductor 44 may protrude above the surface of the transducer 28 just proximal to the transducer 28 to make the electrical connection. Minimizing the exposed central conductor 44 may maximize the protection of the central conductor 44 and the connection between the central conductor 44 and the transducer 28. In some embodiments, the exposed portion of the central conductor 44 may include surface texturing or roughening to increase adhesion between the central conductor 44 and the non-conductive potting adhesive.

FIG. 23 illustrates an alternative bend configuration for the coaxial cable 42. A distal end region of the central conductor 44, the annular conductive member 46, and the dielectric layer 48 may be curved or bent to position the distal end 47 of the central conductor 44 above the transducer 28. This may be performed prior to inserting the drive cable 24 and coaxial cable 42 assembly into the housing 400. In some examples, the central conductor 44, the annular conductive member 46, and the dielectric layer 48 may be bent into the desired shape prior to assembling the coaxial cable 42 with the drive cable 24. However, this is not required. To bend the coaxial cable 42 into the desired shape, the jacket 50, annular conductive member 46, and dielectric layer 48 may be stripped or laser removed from the coaxial cable 42 from the distal end 47 to a location proximal to the transducer 28. The exposed portion of the central conductor 44 (e.g., free from the dielectric layer 48), a distal end region of the annular conductive member 46, and a distal end region of the dielectric layer 48 may be placed into a die and bent into the desired configuration. The angled lead-in of the housing 400 may help guide the pre-bent central conductor 44 within the housing 400.

A distalmost end region 43 of the central conductor 44 may extend generally parallel to the planar inner surface 424. A proximal region 49 of the central conductor 44 may extend generally parallel to the longitudinal axis 401 of the housing 400. The proximal region 49 and the distalmost end region 43 may be interconnected by an intermediate region 51. The intermediate region 51 may extend at a non-parallel angle relative to the planar inner surface 424 and/or the longitudinal axis 401 of the housing 400. In some embodiments, the intermediate region 51 may extend at angle of about 45° to the longitudinal axis 401 of the housing 400. The intermediate region 51 may extend at other non-parallel angles relative to the longitudinal axis 401 of the housing 400, such as, but not limited to, in the range of about 30° to 70° relative to the longitudinal axis 401 of the housing 400. For example, the central conductor 44 may have a more gentle or gradual curve (relative to the abrupt transition of FIG. 22) adjacent to the transducer 28. It is contemplated that bending the annular conductive member 46 and the dielectric layer 48 may allow more of the central conductor 44 to remain covered by the dielectric layer 48 while still positioning the distalmost end region 43 adjacent to the connection point 60′ of the transducer 28. In FIG. 23, the connection point 60′ is a region where the matching layer 36 has been removed to expose the piezoelectric layer 34.

While FIG. 23 does not illustrate the electrical connections between the coaxial cable 42 and the transducer 28/housing 400, it should be understood that these connections are made to complete the electrical circuit. For example, the annular conductive member 46 may be electrically coupled to the housing via solder or ECA, as shown and described with respect to FIG. 3 (e.g., 74 in FIG. 3). Additionally, the central conductor 44 may be electrically coupled to the connection point 60′ with solder or ECA (e.g., 62 in FIG. 3), as shown and described with respect to FIG. 3. After the electrical connections are made, a non-conductive potting adhesive (e.g., 72 in FIG. 3) may be dispensed over the exposed central conductor 44 and the distal end region of the dielectric layer 48. In some cases, the non-conductive potting adhesive may be dispensed over the distal end region of the annular conductive member 46. The non-conductive potting adhesive may grip directly onto the exposed central conductor 44 and cover portions of it. This may protect the central conductor 44 from tensile forces as well as abrasion. For example, the non-conductive potting adhesive may help prevent the central conductor 44 from shifting within the coaxial cable 42. For example, shifting of the central conductor 44 may create tension in the central conductor 44 which may put stress on the connection between the central conductor 44 and the transducer 28. Further, the bent shape of the central conductor 44 may position the central conductor 44 such that most of the exposed central conductor 44 is covered and protected by the non-conductive potting adhesive. The central conductor 44 may protrude above the surface of the transducer 28 just proximal to the transducer 28 to make the electrical connection. Minimizing the exposed central conductor 44 may maximize the protection of the central conductor 44 and the connection between the central conductor 44 and the transducer 28. In some embodiments, the exposed portion of the central conductor 44 may include surface texturing or roughening to increase adhesion between the central conductor 44 and the non-conductive potting adhesive.

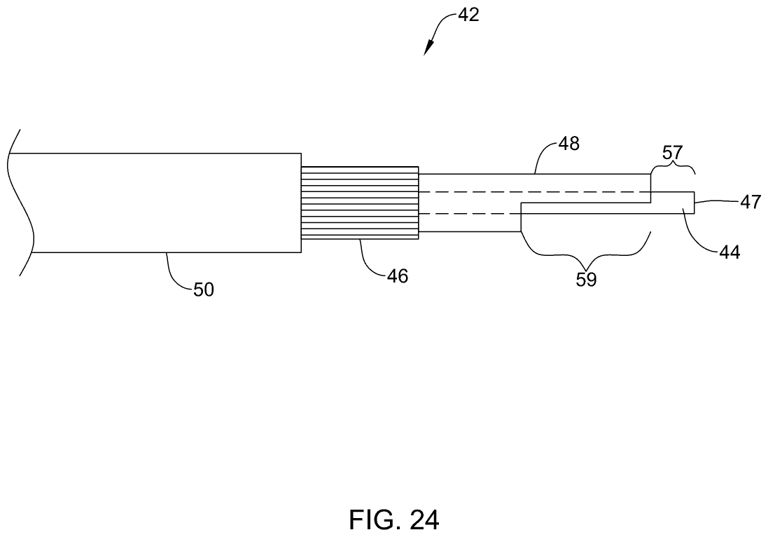

FIG. 24 is a side view of an alternative configuration for the coaxial cable 42. In FIG. 24, the dielectric layer 48 has been laser stripped from the central conductor 44 for a first length 57 extending proximally from the distal end 47 of the central conductor 44. Over the first length 57, the dielectric layer 48 may be removed from an entirety of the circumference of the central conductor 44. This may allow the electrical connection to be made between the central conductor 44 and the transducer 28, 450 by dispensing an electrically conductive material over the distal end region of the central conductor 44, as described herein. Over a second length 59 extending proximally from a proximal end of the first length 57, the dielectric layer 48 may be removed from less than an entirety of the circumference of the central conductor 44. For example, the dielectric layer 48 may be removed from about half (or about 180°) of the circumference of the central conductor 44. However, the dielectric layer 48 may be removed from more than half or less than half of the of the circumference of the central conductor 44, as desired. It is contemplated that the portion of the dielectric layer 48 that is removed over the second length 59 may face towards the lower portion of the housing 26, 250, 300, 400 (e.g., wall 133, 418). Removing only a portion of the dielectric layer 48 may decrease manufacturing time while the residual dielectric layer 48 along the second length 59 may provide additional strength to the central conductor 44. It is contemplated that the central conductor 44 may be adhered to the dielectric layer 48 using a non-conductive potting adhesive, as described herein. Alternatively, or additionally, the central conductor 44 may be adhered to the dielectric layer 48 using extrusion or other bonding techniques.

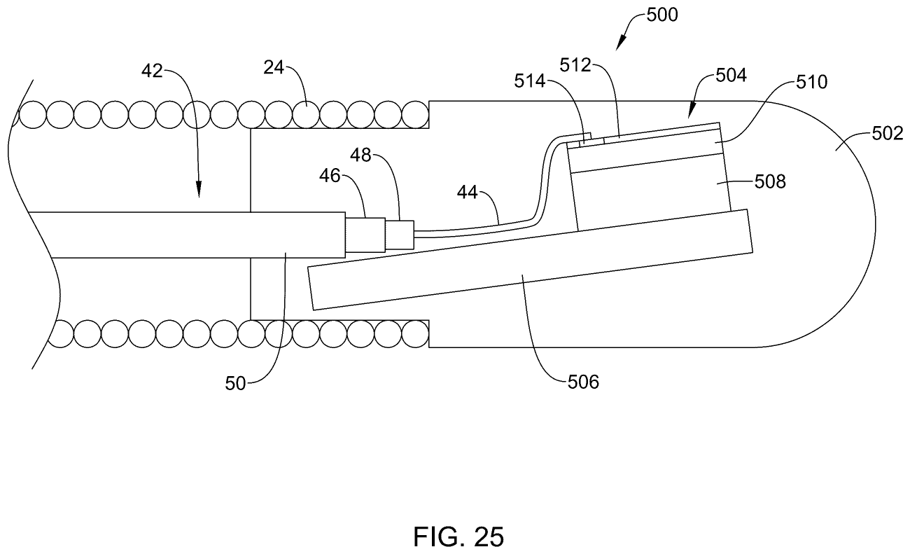

FIG. 25 is a schematic cross-sectional view of another illustrative housing and transducer assembly 500 that may be used with an imaging assembly. The assembly 500 may include a housing 502 formed from an overmolded polymer, a transducer 504, and a circuit board 506. The assembly 500 may be coupled or secured to a drive cable 24. The transducer 504 may be similar in form and function to any of the transducers 28, 450 described herein. For example, the transducer 504 may include a backing layer 508, a piezoelectric layer 510, and a matching layer 512. An electrical connection point 514 may be formed by ablating or removing a region of the matching layer 512. Alternatively, or additionally, the electrical connection point 514 may include a “pre-tin” solder applied before the matching layer 512 with the matching layer 512 removed to expose the pre-tin solder. While not explicitly shown, the transducer 504 may include ledges similar in form and function to ledges 480, 482 described with respect to FIG. 15.

The transducer 504 may be assembled on the circuit board 506 prior to over-modeling the housing 502. The circuit board 506 may electrically couple the bottom surface of the piezoelectric layer 510 with the annular conductive member 46 to form the negative electrical connection. The central conductor 44 may extend to the connection point 514 and may be electrically coupled to an upper surface of the piezoelectric layer 510 to form the positive electrical connection. It is contemplated that any of the coupling mechanisms or techniques described herein may be used to couple the central conductor 44 with the transducer 504. In some embodiments, the central conductor 44 may rest on a ledge similar in form and function to ledge 480 described with respect to FIG. 15 and an ECA may couple the connection point 514 to the central conductor 44 in a manner similar to that described with respect to FIGS. 17-18. In yet another embodiment, a wire bond (not explicitly shown) may extend between the circuit board 506 and an ECA electrically coupled to the connection point 514. The circuit board 506 may be electrically coupled to the central conductor 44 to complete the positive electrical circuit.

Once the electrical connections between the circuit board 506, the coaxial cable 42, and the transducer 504 are complete, the circuit board 506, the distal end region of the coaxial cable 42, and the transducer 504 are over-molded with a polymer to form the housing 502. It is contemplated that the polymer may be an epoxy, a silicone, polyethylene, nylon, acrylonitrile butadiene styrene, or the like. In cases where the housing 502 covers the active face of the transducer 504, the speed of sound in the housing 502 may be lower than the speed of sound in the surrounding medium so that the curved geometry focuses the ultrasound wave. It is contemplated that the matching layer 512 may be formed from a material having a higher acoustic impedance that the material forming the housing 502. Alternatively, the matching layer 512 may be masked during the over-molding process to prevent the polymer housing 502 from covering the matching layer 512.

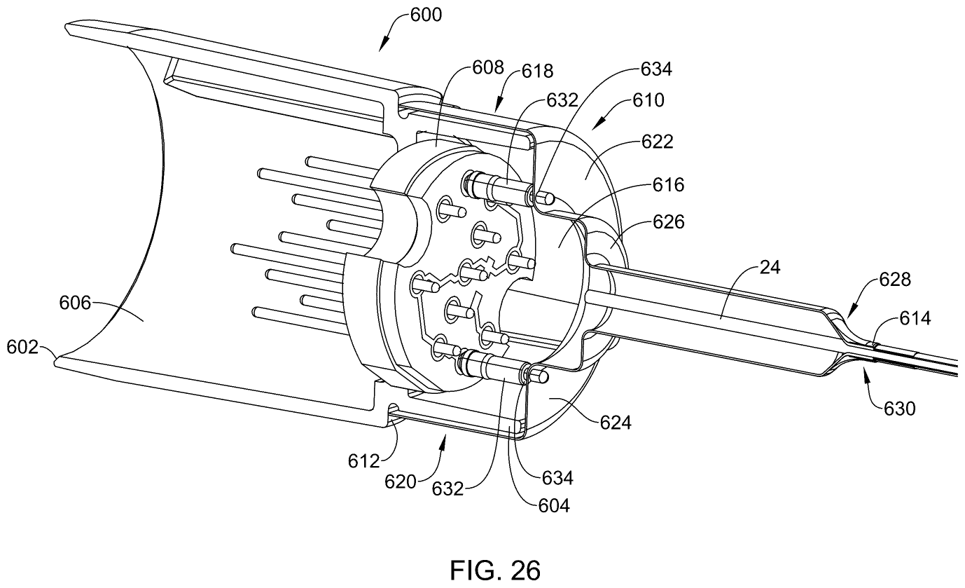

The connector assembly 15 of the medical device 10 may be connected to a drive motor unit (not explicitly shown) in a manner that permits the rotation and translation of the imaging core 22 as well as the ability to power/energize the transducer 28. FIG. 26 is a partial cross-sectional view of a hub 600 that is disposed within the connector assembly 15 (not explicitly shown) and coupled with the drive cable 24. Generally, the hub 600 allows the proximal end of the drive cable 24 to be connected to the negative side of the circuit without manually attaching a wire. Manually attaching a wire may be labor intensive and generate scrap due to variation in the manual process.

The hub 600 may extend from an open proximal end 602 to an open distal end 604. The hub 600 may be generally tubular defining a cavity 606 extending from the proximal end 602 to the distal end 604 thereof. An interface plate 608, which may include a printed circuit board, may be disposed within the cavity 606. The printed circuit board 608 may be coupled to the drive shaft 24 and include electrical connections with the conductor 42 (e.g., and/or the imaging core 22 and/or the drive shaft 24).

An electrically conductive connector shaft 610 may extend from a proximal end 612 to a distal end 614. In some embodiments, the connector shaft 610 may be formed from stainless steel. However, the connector shaft 610 may be formed from other electrically conductive materials. The connector shaft 610 may define a cavity 616 therein. A proximal end region 618 may be disposed over and mechanically secured to a distal end region 620 of the hub 600. The connector shaft 610 may have a first inner diameter adjacent to the proximal end 612 thereof and a second, smaller, inner diameter adjacent the distal end 614 thereof. The first inner diameter may be substantially the same as an outer diameter of the distal end region 620 of the hub 600. However, this is not required. The diameter of the connector shaft 610 may reduce from the first diameter to the second diameter in increments along a length thereof. A first transition 622 in inner diameter may occur adjacent to the distal end 604 of the hub 600. At the first transition 622, the side wall 624 of the connector shaft 610 may extend generally orthogonal to a longitudinal axis of the hub 600 such that the side wall 624 at least partially occludes the distal opening at the distal end 604 of the hub 600. Two or more additional transitions 626, 628 in the inner diameter may occur between the first transition 622 and the distal end 614 of the connector shaft 610. The two or more transitions 626, 628 may be abrupt or gradual, as desired. Further, the connector shaft 610 may include fewer than three or more than three transition in inner diameter, as desired. The flat surface of the medium diameter transition 626 may serve as a thrust bearing surface as the connector is pushed distally when it is connected to the motor drive unit. The small diameter transition 628 may serve as a seal to contain saline or other fluid within the catheter.

A distal end region 630 of the connector shaft 610 may be secured to an outer surface of the drive cable 24. The drive cable 24 may be mechanically coupled to the connector shaft 610 with a torsionally strong joint. In some cases, the distal end region 630 may be crimped, soldered, welded, adhered, combinations thereof, etc. to the drive cable 24. It is contemplated that heat-based processes such as soldering or welding may require minimization of heat transfer to the coaxial cable 42 to avoid melting portions of the coaxial cable 42. It is further contemplated that UV cured adhesives, electrically conductive adhesives, non-conductive adhesives, or the like may be used.

Pogo pins 632 may extend from the printed circuit board 608. The pogo pins 632 may electrically couple the negative side of the circuit to drive cable 24 via the connector shaft 610. The pogo pins 632 may extend through apertures 634 in the side wall 624 to electrically couple the printed circuit board 608 with the connector shaft 610.

The materials that can be used for the various components of the system 10 (and/or other systems disclosed herein) may include those commonly associated with medical devices. For simplicity purposes, the following discussion makes reference to the shaft 12 and other components of the system 10. However, this is not intended to limit the devices and methods described herein, as the discussion may be applied to other similar tubular members and/or components of tubular members or devices disclosed herein.

The shaft 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some cases, the shaft 12 and/or other components of the system 10 may include polymeric coatings for fillers. Some examples for suitable polymers for coating or fillers may include, but are not limited to, parylene (poly-para-xylylene), poly-dimethyl siloxane (PDMS), poly-methyl methacrylate (PMMA), and poly-(vinylidene fluoride) (PVDF), polyacrylonitrile (PAN), epoxy resins, or the like.

Adhesives or electrically conductive adhesives may be used in the coupling of various components of the shaft and/or other components of the system 10. Electrically conductive adhesives may include a conductive component, such as, but not limited to iron, silver, copper, nickel, graphite, or the like, suspended in an adhesive. Some examples of adhesives include acrylics, epoxies, urethanes, hydrocolloids, hydrogels, cyanoacrylates, silicones, or the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof, and the like; or any other suitable material.

In at least some embodiments, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging device, comprising:
a shaft having a proximal end region and a distal end region;
an imaging assembly movably disposed within the shaft, the imaging assembly comprising:
a drive cable;
a housing coupled to a distal end of the drive cable;
a transducer disposed within the housing; and
a coaxial cable extending through the drive cable and electrically coupled to the transducer;
wherein the coaxial cable is electrically coupled to a connection point on an upper surface of the transducer and electrically coupled to the housing.

2. The intravascular imaging device of claim 1, wherein the connection point on the upper surface of the transducer is a pre-tin solder.

3. The intravascular imaging device of claim 1, wherein a first member of the coaxial cable is electrically coupled to the connection point.

4. The intravascular imaging device of claim 3, wherein a distal end region of the first member is bent to align the distal end region of the first member with the connection point.

5. The intravascular imaging device of claim 4, wherein a second member of the coaxial cable is electrically coupled to the housing.

6. The intravascular imaging device of claim 5, wherein the first and second members of the coaxial cable are separated by a dielectric layer.

7. The intravascular imaging device of claim 4, wherein the second member of the coaxial cable is electrically coupled to the housing with an electrically conductive adhesive or solder.

8. The intravascular imaging device of claim 1, wherein the housing comprises a generally tubular proximal end region, an intermediate region having a cavity for receiving the transducer, and a distal end region having a substantially solid cross-section.

9. The intravascular imaging device of claim 8, wherein the distal end region of the housing further comprises a notch that is circumferentially aligned with an active surface of the transducer.

10. The intravascular imaging device of claim 1, further comprising a non-conductive potting adhesive disposed over an exposed portion of a first member of the coaxial cable.

11. The intravascular imaging device of claim 8, wherein the proximal end region of the housing includes a cut-out region extending through a sidewall of the housing, the cut-out region extending proximally from the intermediate region of the housing.

12. An intravascular imaging device, comprising:
a shaft having a proximal end region and a distal end region; and
an imaging assembly movably disposed within the shaft, the imaging assembly comprising:
a drive cable;
a housing coupled to a distal end of the drive cable, the housing comprising:
a proximal end region;
an intermediate region having a cavity for receiving a transducer; and
a distal end region having a substantially solid cross-section;
a transducer disposed within the cavity of the housing, the transducer comprising:
a backing layer extending from a proximal end to a distal end, the backing layer comprising at least a first portion having a first length and a second portion having a second length, the first portion extending proximally beyond a proximal end of the second portion to define a ledge;
a piezoelectric layer disposed on the backing layer;
a matching layer disposed on the piezoelectric layer, the matching layer extending across an upper surface of the piezoelectric layer, the ledge of the backing layer, and at least some of the lateral and axial sides of the piezoelectric layer and the backing layer; and
a coaxial cable extending through the drive cable and electrically coupled to the transducer.

13. The intravascular imaging device of claim 12, wherein a distal end region of a first portion of the coaxial cable is positioned on the ledge of the backing layer.

14. The intravascular imaging device of claim 12, wherein a region of the matching layer is removed to define a connection point adjacent to the proximal end of the transducer.

15. The intravascular imaging device of claim 14, further comprising an electrically conductive adhesive extending between the connection point and the ledge of the backing layer, the electrically conductive adhesive configured to electrically couple the connection point with the coaxial cable.

16. An intravascular imaging device, comprising:
a shaft having a proximal end region and a distal end region;
an imaging assembly movably disposed within the shaft, the imaging assembly comprising:
a drive cable;
a housing coupled to a distal end of the drive cable;
a transducer disposed within the housing;
a coaxial cable extending through the drive cable and electrically coupled to the transducer; and
a connector assembly coupled to a proximal end of the drive cable, the connector assembly comprising:
a hub having a cavity;
a printed circuit board disposed within the cavity; and
an electrically conductive connector shaft coupled to the drive cable and electrically connected to the printed circuit board.

17. The intravascular imaging device of claim 16, wherein the electrically conductive connector shaft comprises a proximal end region disposed over a distal end region of the hub.

18. The intravascular imaging device of claim 16, wherein the electrically conductive connector shaft comprises a distal end region secured to an outer surface of the drive cable.

19. The intravascular imaging device of claim 16, further comprising one or more pogo pins extending from the printed circuit board and electrically coupling the printed circuit board with the connector shaft.

20. The intravascular imaging device of claim 16, wherein the connector shaft comprises a first inner diameter adjacent to a proximal end and a second, smaller inner diameter adjacent to a distal end.
